(19) United States
(12) Patent Application Publication
(10) Pub. No.: US 2026/0048259 A1
(43) Pub. Date: Feb. 19, 2026

(54) SUBSTANCE DELIVERY INSIDE MAMMALS

(71) Applicant: COCHLEAR LIMITED, Macquarie University, NSW (AU)

(72) Inventors: Daniel SMYTH, Macquarie University (AU); Wolfram Frederik DUECK, Macquarie University (AU)

(21) Appl. No.: 19/104,084

(22) PCT Filed: Aug. 19, 2023
(86) PCT No.: PCT/IB2023/058306

§ 371 Date: Feb. 14, 2025

Related U.S. Application Data
(60) Provisional application No. 63/399,613, filed on Aug. 19, 2022.

(51) Int. Cl. A61N 1/05; A61M 5/142; A61N 1/36

(52) U.S. Cl. CPC A61N 1/0541; A61M 5/14276; A61N 1/36038; A61M 2202/0007; A61M 2205/04; A61M 2210/0662

(57) ABSTRACT

An apparatus, including an array of electrodes and an implantable therapeutic substance reservoir, wherein the apparatus is an implantable portion of a cochlear implant, the apparatus is configured so that the therapeutic substance reservoir extends from a location behind an ear canal of a human between a mastoid bone and skin of the human to the cochlea when the apparatus is fully implanted in a recipient, and the reservoir is completely integrated into the implantable portion of the cochlear implant.

### CROSS-REFERENCE TO RELATED APPLICATIONS

This application claims priority to U.S. Provisional Application No. 63/399,613, entitled SUBSTANCE DELIVERY INSIDE MAMMALS, filed on Aug. 19, 2022, naming Daniel SMYTH as an inventor, the entire contents of that application being incorporated herein by reference in its entirety.

### BACKGROUND

Medical devices have provided a wide range of therapeutic benefits to recipients over recent decades. Medical devices can include internal or implantable components/devices, external or wearable components/devices, or combinations thereof (e.g., a device having an external component communicating with an implantable component). Medical devices, such as traditional hearing aids, partially or fully-implantable hearing prostheses (e.g., bone conduction devices, mechanical stimulators, cochlear implants, etc.), pacemakers, defibrillators, functional electrical stimulation devices, and other medical devices, have been successful in performing lifesaving and/or lifestyle enhancement functions and/or recipient monitoring for a number of years.

The types of medical devices and the ranges of functions performed thereby have increased over the years. For example, many medical devices, sometimes referred to as “implantable medical devices,” now often include one or more instruments, apparatus, sensors, processors, controllers or other functional mechanical or electrical components that are permanently or temporarily implanted in a recipient. These functional devices are typically used to diagnose, prevent, monitor, treat, or manage a disease/injury or symptom thereof, or to investigate, replace or modify the anatomy or a physiological process. Many of these functional devices utilize power and/or data received from external devices that are part of, or operate in conjunction with, implantable components.

### SUMMARY

In an exemplary embodiment, there is an apparatus, comprising an array of electrodes and an implantable therapeutic substance reservoir, wherein the apparatus is an implantable portion of a cochlear implant, the apparatus is configured so that the therapeutic substance reservoir extends from a location behind an ear canal of a human between a mastoid bone and skin of the human to the cochlea when the apparatus is fully implanted in a recipient, and the reservoir is completely integrated into the implantable portion of the cochlear implant.

In an exemplary embodiment, there is an apparatus, comprising: an array of electrodes; and an implantable therapeutic substance reservoir, wherein the apparatus is an implantable portion of a cochlear implant, the apparatus is configured so that the therapeutic substance reservoir extends from a location behind an ear canal of a human between a mastoid bone and skin of the human to the cochlea when the apparatus is fully implanted in a recipient, and the reservoir is completely integrated into the implantable portion of the cochlear implant.

In an exemplary embodiment, there is an apparatus, comprising: an implantable therapeutic substance reservoir; a plurality of implantable electrodes; an implantable silicone carrier body supporting the plurality of electrodes; and a stimulator assembly including an implantable housing and stimulation electronics, wherein the therapeutic substance reservoir extends from the stimulator assembly to the silicone carrier body, and the apparatus includes a fill port in fluid communication with the reservoir configured to enable therapeutic substance delivery to the reservoir, and the fill port is between the housing and the plurality of electrodes.

In an exemplary embodiment, there is a method, comprising: obtaining a sterilized sealed package containing an implantable portion of a hearing prosthesis including an empty therapeutic substance delivery system; and providing therapeutic substance to the therapeutic substance delivery system while the implantable portion is supported by at least a portion of the package.

In an exemplary embodiment, there is an apparatus, comprising: an implantable electrode array including a plurality of electrodes supported by a silicone body, wherein the electrode array includes a therapeutic substance delivery channel including at least one port, and port includes a distinct barrier made of a material that maintains a bacterial barrier prior to charging the channel with a water-based substance.

In an exemplary embodiment, there is an implantable medical device, comprising: an implantable therapeutic substance reservoir including a cistern and tube(s) extending from the cistern and in fluid communication with the cistern; a plurality of implantable electrodes made of a biocompatible metal connected to or integral with respective electrical leads, respective electrodes of the plurality of electrodes being spatially separated from one another; an implantable elongate silicone carrier body supporting the plurality of electrodes and encasing at least partially the respective electrical leads; and a stimulator unit in electrical communication with the respective electrical leads, the stimulator unit including an implantable housing and electronics that are configured to transform a received signal into an output signal for output to one or more of the electrodes via the respective electrical leads, wherein the tube(s) of the therapeutic substance reservoir extend from the stimulator unit to the silicone carrier body and extend in the silicone carrier body, and the implantable medical device includes a fill port in fluid communication with the reservoir configured to enable therapeutic substance delivery to the reservoir, and the fill port is between the housing and the plurality of electrodes.

### BRIEF DESCRIPTION OF THE DRAWINGS

Embodiments are described below with reference to the attached drawings, in which:

FIG. 1A is a perspective view of an exemplary hearing prosthesis in which at least some of the teachings detailed herein are applicable;

FIGS. 1B-1D are quasi functional diagrams of an exemplary device to which some embodiments may be applicable;

FIGS. 1E and 2A and 2B and 2C and 1F present some schematics related to base technologies associated with some embodiments;

FIGS. 3 and 4 show other exemplary medical devices to which at least some of the teachings herein are applicable;

FIGS. 5 and 5A show top views of exemplary implantable portions of a cochlear implant according to an embodiment;

FIGS. 6 and 7 show some details of a portion of an exemplary implantable portion of a cochlear implant according to an embodiment;

FIG. 8 shows a side view of an ear for reference purposes;

FIGS. 9-11 show some details of some portions of an exemplary implantable portion of a cochlear implant according to an embodiment;

FIGS. 12 and 13 show an exemplary implantable portion of a cochlear implant and a reservoir fill assembly unattached and attached to each other;

FIG. 14 shows some details of a portion of an exemplary implantable portion of a cochlear implant according to an embodiment;

FIG. 15 shows an exemplary reservoir fill assembly;

FIG. 16 shows an exemplary flowchart for an exemplary algorithm;

FIGS. 17 and 18 show exemplary assemblies that include packaging; and

FIGS. 18A-20 show some details of some portions of exemplary implantable portions of a cochlear implant according to an embodiment.

### DETAILED DESCRIPTION

Merely for ease of description, the techniques presented herein are primarily described herein with reference to an illustrative medical device, namely a hearing prosthesis. First introduced is a cochlear implant. The techniques presented herein may also be used with a variety of other medical devices that, while providing a wide range of therapeutic benefits to recipients, patients, or other users, may benefit from the teachings herein used in other medical devices. For example, any techniques presented herein described for one type of hearing prosthesis, such as a cochlear implant, corresponds to a disclosure of another embodiment of using such teaching with, at least in conjunction with, another hearing prosthesis, including bone conduction devices (percutaneous, active transcutaneous and/or passive transcutaneous), middle ear auditory prostheses, direct acoustic stimulators, and also utilizing such with other electrically simulating auditory prostheses (e.g., auditory brain stimulators), etc. The techniques presented herein can be used with implantable/implanted microphones, whether or not used as part of a hearing prosthesis (e.g., a body noise or other monitor, whether or not it is part of a hearing prosthesis) and/or external microphones. The techniques presented herein can also be used with vestibular devices (e.g., vestibular implants), sensors, seizure devices (e.g., devices for monitoring and/or treating epileptic events, where applicable), sleep apnea devices, retinal implants, electroporation, etc., and thus any disclosure herein is a disclosure of utilizing such devices with the teachings herein, providing that the art enables such.

Note also embodiments include the application of the teachings herein to a medical device that is a non-implanted medical device, such as a minimally invasive probe used by medical personnel.

By way of example, any of the technologies detailed herein which are associated with components that are implanted in a recipient can be combined with information delivery technologies disclosed herein, such as for example, devices that evoke a hearing percept, to convey information to the recipient. By way of example only and not by way of limitation, a sleep apnea implanted device can be combined with a device that can evoke a hearing percept so as to provide information to a recipient, such as status information, etc. In this regard, the various sensors detailed herein and the various output devices detailed herein can be combined with such a non-sensory prosthesis or any other nonsensory prosthesis that includes implantable components so as to enable a user interface, as will be described herein, that enables information to be conveyed to the recipient, which information is associated with the implant.

While the teachings detailed herein will be described for the most part with respect to hearing prostheses, in keeping with the above, it is noted that any disclosure herein with respect to a hearing prosthesis corresponds to a disclosure of another embodiment of utilizing the associated teachings with respect to any of the other prostheses noted herein, whether a species of a hearing prosthesis, or a species of a sensory prosthesis.

The techniques presented herein are also described with reference by way of background to another illustrative medical device, namely a retinal implant. As noted above, the techniques presented herein are also applicable to the technology of vestibular devices (e.g., vestibular implants), visual devices (i.e., bionic eyes), as well as sensors, pacemakers, drug delivery systems, defibrillators, functional electrical stimulation devices, catheters, seizure devices (e.g., devices for monitoring and/or treating epileptic events), sleep apnea devices, electroporation, etc.

Any reference to one of the above-noted sensory prostheses corresponds to an alternate disclosure using one of the other above-noted sensory prostheses unless otherwise noted providing that the art enables such.

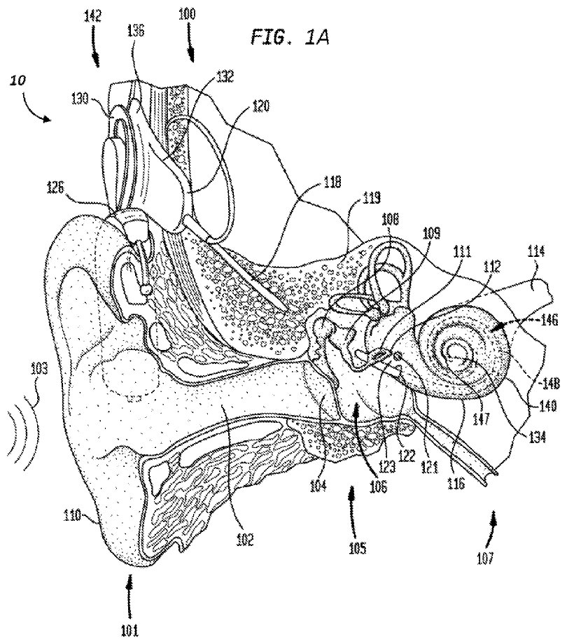

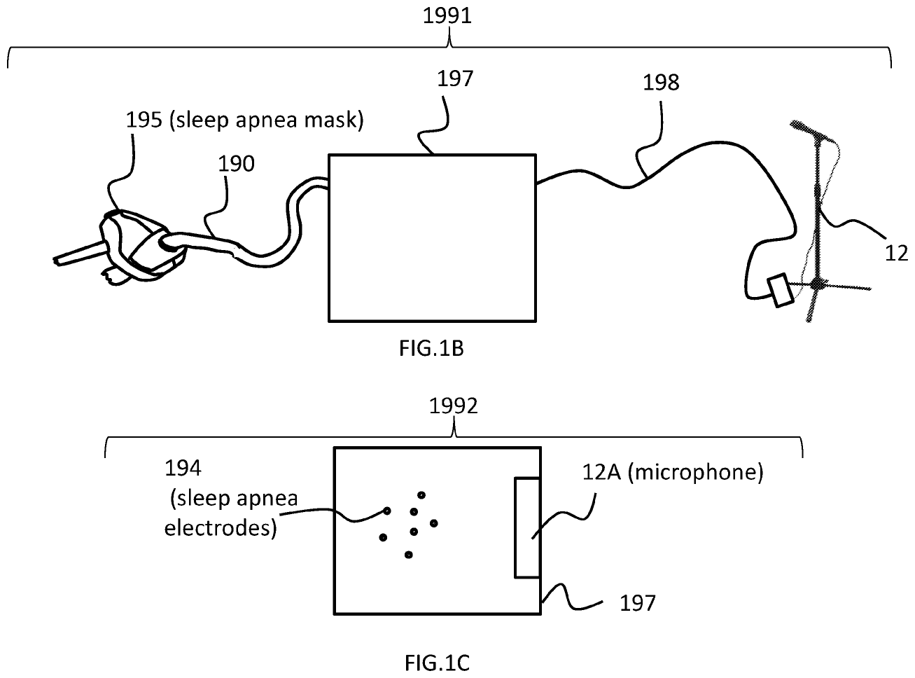

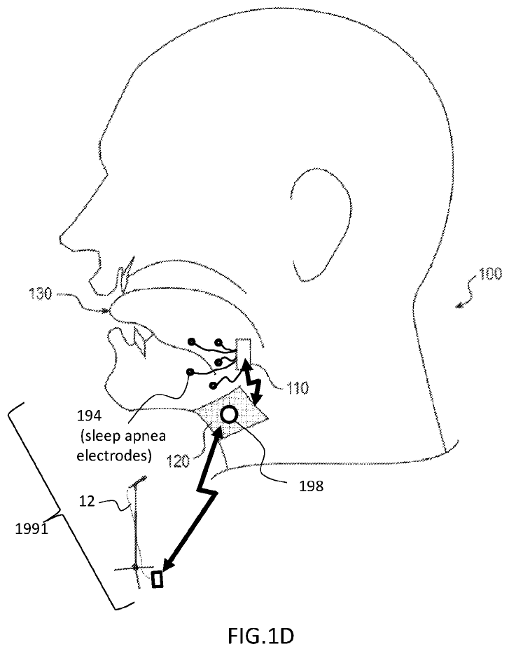

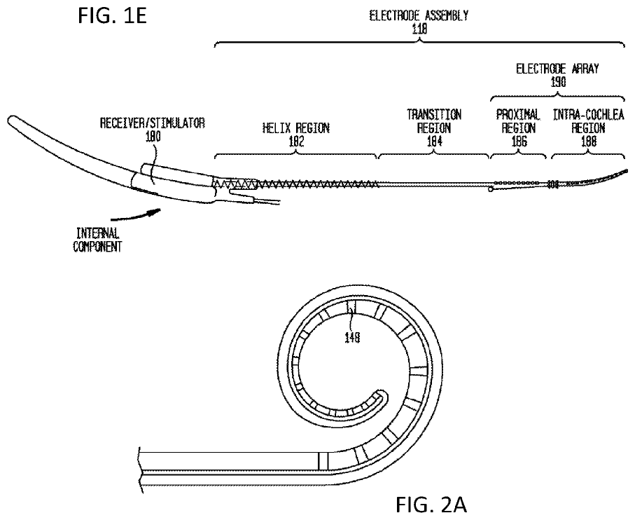

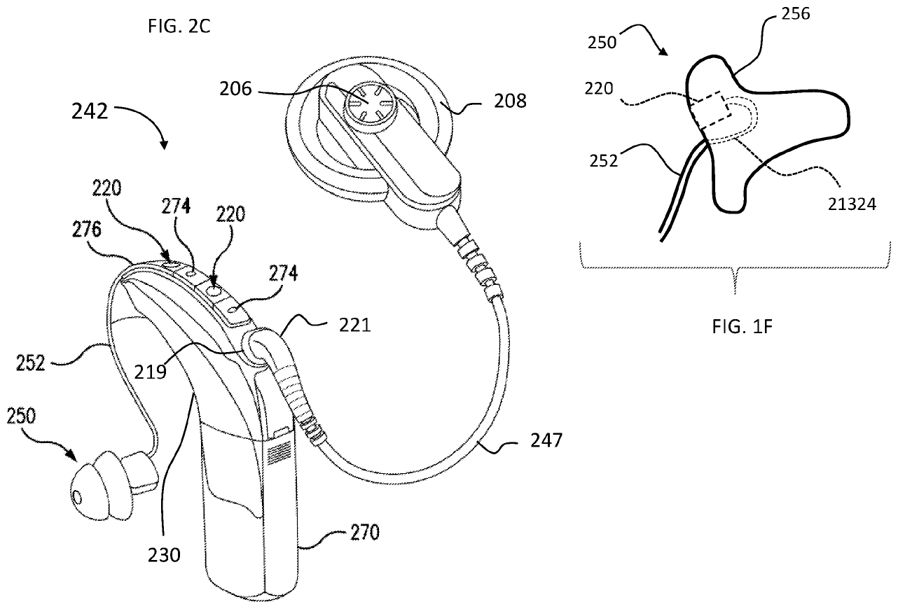

FIG. 1A is perspective view of an implantable portion of a cochlear implant 100, implanted in a recipient. The implantable portion of the cochlear implant 100 is part of a partially implantable cochlear implant system 10 that can include external component(s), as will be detailed below.

The recipient has an outer ear 101, a middle ear 105, and an inner ear 107. Components of outer ear 101, middle ear 105, and inner ear 107 are described below, followed by a description of implant 100.

In a fully functional ear, outer ear 101 comprises an auricle 110 and an ear canal 102. An acoustic pressure or sound wave 103 is collected by auricle 110 and channeled into and through ear canal 102. Disposed across the distal end of ear canal 102 is a tympanic membrane 104 which vibrates in response to sound wave 103. This vibration is coupled to oval window or fenestra ovalis 112 through three bones of middle ear 105, collectively referred to as the ossicles 106 and comprising the malleus 108, the incus 109, and the stapes 111. Bones 108, 109, and 111 of middle ear 105 serve to filter and amplify sound wave 103, causing oval window 112 to articulate, or vibrate in response to vibration of tympanic membrane 104. This vibration sets up waves of fluid motion of the perilymph within cochlea 140. Such fluid motion, in turn, activates tiny hair cells (not shown) inside of cochlea 140. Activation of the hair cells causes appropriate nerve impulses to be generated and transferred through the spiral ganglion cells (not shown) and auditory nerve 114 to the brain (also not shown) where they are perceived as sound.

As shown, implantable portion of cochlear implant 100 comprises one or more components which are temporarily or permanently implanted in the recipient. Implant 100 is shown in FIG. 1A with an external device 142, that is part of system 10 (along with implantable portion of the cochlear implant 100), which, as described below, is configured to provide power to the implant.

In the illustrative arrangement of FIG. 1A, external device 142 may comprise a power source (not shown) disposed in a Behind-The-Ear (BTE) unit 126. External device 142 also includes components of a transcutaneous energy transfer link, referred to as an external energy transfer assembly. The transcutaneous energy transfer link is used to transfer power and/or data to implant 100. Various types of energy transfer, such as infrared (IR), electromagnetic, capacitive, and inductive transfer, may be used to transfer the power and/or data from external device 142 to implant 100. In the illustrative embodiments of FIG. 1A, the external energy transfer assembly comprises an external coil 130 that forms part of an inductive radio communication link. External coil 130 is typically a wire antenna coil comprised of multiple turns of electrically insulated single-strand or multi-strand platinum or gold wire. External device 142 also includes a magnet (not shown) positioned within the turns of wire of external coil 130. It should be appreciated that the external device shown in FIG. 1A is merely illustrative, and other external devices may be used with embodiments of the present invention.

Implantable portion of the cochlear implant 100 comprises an internal energy transfer assembly 132 which may be positioned in a recess of the temporal bone adjacent auricle 110 of the recipient. As detailed below, internal energy transfer assembly 132 is a component of the transcutaneous energy transfer link and receives power and/or data from external device 142. In the illustrative embodiment, the energy transfer link comprises an inductive RF link, and internal energy transfer assembly 132 comprises a primary internal coil 136. Internal coil 136 is typically a wire antenna coil comprised of multiple turns of electrically insulated single-strand or multi-strand platinum or gold wire.

The implantable portion of the cochlear implant 100 further comprises a main implantable component 120 and an elongate stimulating assembly 118. In embodiments of the present invention, internal energy transfer assembly 132 and main implantable component 120 are hermetically sealed within a biocompatible housing. In embodiments of the present invention, main implantable component 120 includes a sound processing unit (not shown) to convert the sound signals received by the implantable microphone in internal energy transfer assembly 132 to data signals. Main implantable component 120 further includes a stimulator unit (also not shown) which generates electrical stimulation signals based on the data signals. The electrical stimulation signals are delivered to the recipient via elongate stimulating assembly 118.

Elongate stimulating assembly 118 has a proximal end connected to main implantable component 120, and a distal end implanted in cochlea 140. Stimulating assembly 118 extends from main implantable component 120 to cochlea 140 through mastoid bone 119. In some embodiments stimulating assembly 118 may be implanted at least in basal region 116, and sometimes further. For example, stimulating assembly 118 may extend towards apical end of cochlea 140, referred to as cochlea apex 134. In certain circumstances, stimulating assembly 118 may be inserted into cochlea 140 via a cochleostomy 122. In other circumstances, a cochleostomy may be formed through round window 121, oval window 112, the promontory 123 or through an apical turn 147 of cochlea 140.

Stimulating assembly 118 comprises a longitudinally aligned and distally extending array 146 of electrodes 148, disposed along a length thereof. As noted, a stimulator unit generates stimulation signals which are applied by stimulating contacts 148, which, in an exemplary embodiment, are electrodes, to cochlea 140, thereby stimulating auditory nerve 114. In an exemplary embodiment, stimulation contacts can be any type of component that stimulates the cochlea (e.g., mechanical components, such as piezoelectric devices that move or vibrate, thus stimulating the cochlea (e.g., by inducing movement of the fluid in the cochlea), electrodes that apply current to the cochlea, etc.). Embodiments detailed herein will generally be described in terms of an electrode assembly 118 utilizing electrodes as elements 148. It is noted that alternate embodiments can utilize other types of stimulating devices. Any device, system, or method of stimulating the cochlea via a device that is located in the cochlea can be utilized in at least some embodiments. In this regard, any implantable array that stimulates tissue, such as a retinal implant array, or a spinal array, or a pacemaker array, etc., is encompassed within the teachings herein unless otherwise noted.

As noted, the implantable portion 100 comprises a partially implantable prosthesis, as contrasted to a totally implantable prosthesis that is capable of operating, at least for a period of time, without the need for external device 142. Therefore, implantable portion of cochlear implant 100 does not comprise a rechargeable power source that stores power received from external device 142, as contrasted to an embodiment where there is an implantable rechargeable power source (e.g., a rechargeable battery). During operation of implant 100, the power is transferred from the external component to the implanted component via the link, and distributed to the various other implanted components as needed.

It is noted that the teachings detailed herein and/or variations thereof can be utilized with a totally implantable prosthesis. That is, in an alternate embodiment of the cochlear implants or other hearing prostheses detailed herein, the prostheses are totally implantable prostheses, such as where there is an implanted microphone and sound processor and battery.

FIG. 1B provides a schematic of an exemplary conceptual sleep apnea system 1991. Here, this exemplary sleep apnea system utilizes a microphone 12 (represented conceptually) to capture a person's breathing or otherwise the sounds made by a person while sleeping. The microphone transduces the captured sound into an electrical signal which is provided via electrical leads 198 to the main unit 197, which includes a processor unit that can evaluate the signal from leads 198 or, in another arrangement, unit 197 is configured to provide that signal to a remote processing location via the Internet or the like, where the signal was evaluated. Upon an evaluation that an action should be taken or otherwise can be utilitarian taken by the sleep apnea system 1991, the unit 197 activates to implement sleep apnea countermeasures, which countermeasures are conducted by a hose 1902 sleep apnea mask 195. By way of example only and not by way of limitation, pressure variations can be used to treat the sleep apnea upon an indication of such an occurrence.

In an exemplary embodiment, the advanced implantation methods and devices detailed herein can be utilized to treat sleep apnea/in a device that can be used to treat. Specifically, the electrodes of the implant disclosed below can be utilized in place of the electrodes 194 (placed accordingly, of course), and the implant can be of a configuration to treat sleep apnea. In this regard, in an exemplary embodiment, the implantable components detailed herein can be located at locations to treat sleep apnea in accordance with the teachings herein, with the requisite modification if necessary or otherwise utilitarian to implement such.

FIGS. 1C and 1D provide another exemplary schematic of another exemplary conceptual sleep apnea system 1992. Here, the sleep apnea system is different from that of FIG. 1B in that electrodes 194 (which can be implanted in some embodiments) are utilized to provide stimulation to the human who is experiencing a sleep apnea scenario. FIG. 1C illustrates an external unit, and FIG. 1D illustrates the external unit 120 and an implanted unit 110 in signal communication via an inductance coil 707 of the external unit and a corresponding implanted inductance coil (not shown) of the implanted unit, according to which the teachings herein can be applicable. Implanted unit 110, can be configured for implantation in a recipient, in a location that permits it to modulate nerves of the recipient 100 via electrodes 194. In treating sleep apnea, implant unit 110 and/or the electrodes thereof can be located on a genioglossus muscle of a patient. Such a location is suitable for modulation of the hypoglossal nerve, branches of which run inside the genioglossus muscle.

External unit 120 can be configured for location external to a patient, either directly contacting, or close to the skin of the recipient. External unit 120 may be configured to be affixed to the patient, for example, by adhering to the skin of the patient, or through a band or other device configured to hold external unit 120 in place. Adherence to the skin of external unit 120 may occur such that it is in the vicinity of the location of implant unit 110 so that, for example, the external unit 120 can be in signal communication with the implant unit 110 as conceptually shown, which communication can be via an inductive link or an RF link or any link that can enable treatment of sleep apnea using the implant unit and the external unit. External unit 120 can include a processor unit 198 that is configured to control the stimulation executed by the implant unit 110. In this regard, processor unit 198 can be in signal communication with microphone 12, via electrical leads, such as in an arrangement where the external unit 120 is a modularized component, or via a wireless system, such as conceptually represented in FIG. 1D.

A common feature of both of these sleep apnea treatment systems is the utilization of the microphone to capture sound, and the utilization of that captured sound to implement one or more features of the sleep apnea system. In some embodiments, the teachings herein are used with the sleep apnea device just detailed.

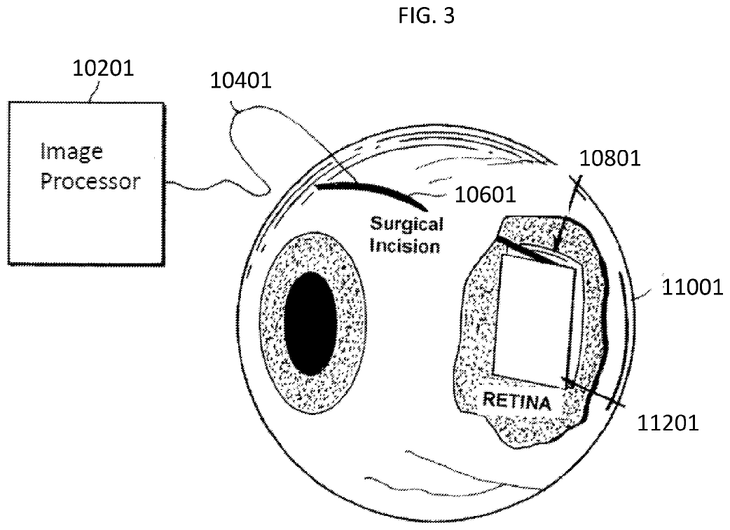

FIG. 3 presents an exemplary embodiment of a neural prosthesis in general, and a retinal prosthesis and an environment of use thereof, in particular, the components of which can be used in whole or in part, with some of the teachings herein. In some embodiments of a retinal prosthesis, a retinal prosthesis sensor-stimulator 10801 is positioned proximate the retina 11001. In an exemplary embodiment, photons entering the eye are absorbed by a microelectronic array of the sensor-stimulator 10801 that is hybridized to a glass piece 11201 containing, for example, an embedded array of microwires. The glass can have a curved surface that conforms to the inner radius of the retina. The sensor-stimulator 108 can include a microelectronic imaging device that can be made of thin silicone containing integrated circuitry that convert the incident photons to an electronic charge.

An image processor 10201 is in signal communication with the sensor-stimulator 10801 via cable 10401 which extends through surgical incision 00601 through the eye wall (although in other embodiments, the image processor 10201 is in wireless communication with the sensor-stimulator 10801). The image processor 10201 processes the input into the sensor-stimulator 10801 and provides control signals back to the sensor-stimulator 10801 so the device can provide processed output to the optic nerve. That said, in an alternate embodiment, the processing is executed by a component proximate with or integrated with the sensor-stimulator 10801. The electric charge resulting from the conversion of the incident photons is converted to a proportional amount of electronic current which is input to a nearby retinal cell layer. The cells fire and a signal is sent to the optic nerve, thus inducing a sight perception.

The retinal prosthesis can include an external device disposed in a Behind-The-Ear (BTE) unit or in a pair of eyeglasses, or any other type of component that can have utilitarian value. The retinal prosthesis can include an external light/image capture device (e.g., located in/on a BTE device or a pair of glasses, etc.), while, as noted above, in some embodiments, the sensor-stimulator 10801 captures light/images, which sensor-stimulator is implanted in the recipient.

In the interests of compact disclosure, any disclosure herein of a microphone or sound capture device corresponds to an analogous disclosure of a light/image capture device, such as a charge-coupled device. Corollary to this is that any disclosure herein of a stimulator unit which generates electrical stimulation signals or otherwise imparts energy to tissue to evoke a hearing percept corresponds to an analogous disclosure of a stimulator device for a retinal prosthesis. Any disclosure herein of a sound processor or processing of captured sounds or the like corresponds to an analogous disclosure of a light processor/image processor that has analogous functionality for a retinal prosthesis, and the processing of captured images in an analogous manner. Indeed, any disclosure herein of a device for a hearing prosthesis corresponds to a disclosure of a device for a retinal prosthesis having analogous functionality for a retinal prosthesis. Any disclosure herein of fitting a hearing prosthesis corresponds to a disclosure of fitting a retinal prosthesis using analogous actions. Any disclosure herein of a method of using or operating or otherwise working with a hearing prosthesis herein corresponds to a disclosure of using or operating or otherwise working with a retinal prosthesis in an analogous manner.

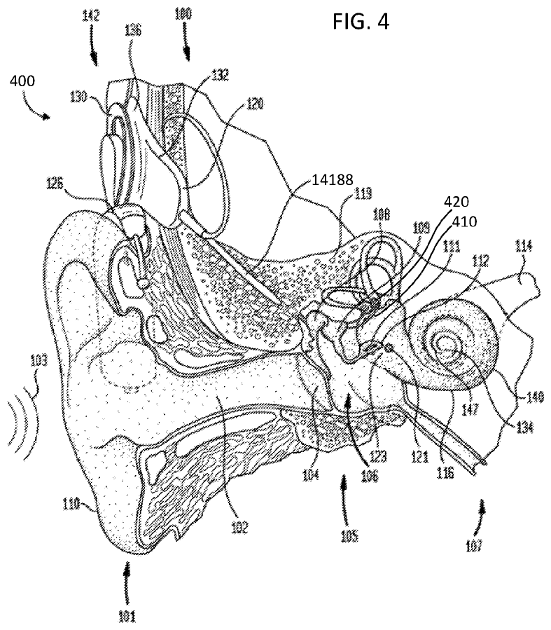

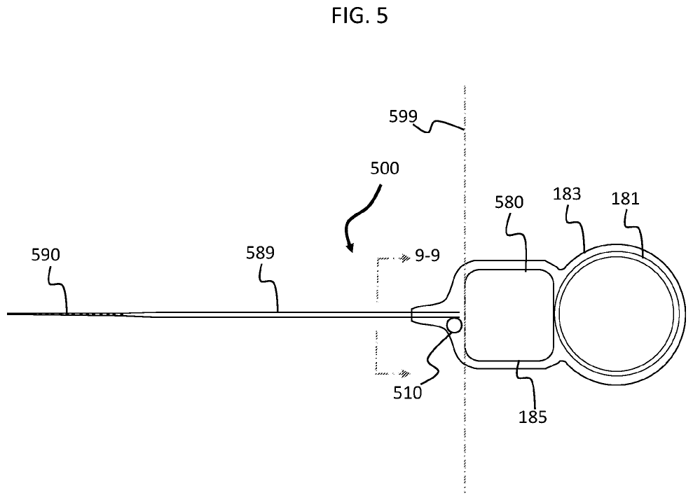

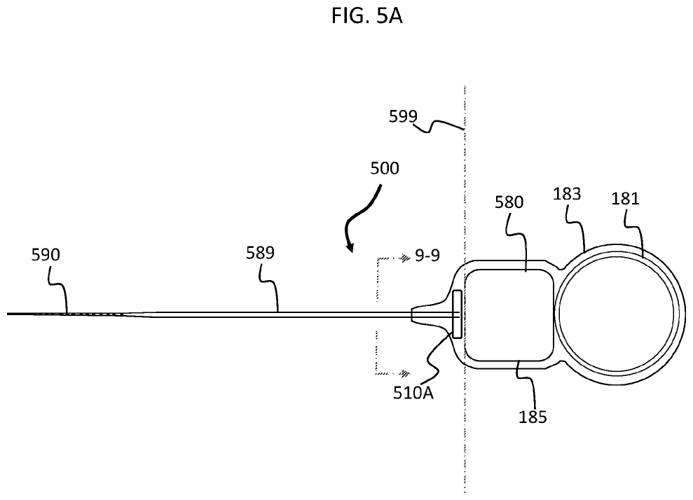

FIG. 4 depicts an exemplary vestibular implant 400 according to one example. Some specific features are described utilizing the above-noted cochlear implant of FIG. 1 in contacts for the various elements. In this regard, some features of a cochlear implant are utilized with vestibular implants. In the interest of textual and pictorial economy, various elements of the vestibular implant that generally correspond to the elements of the cochlear implant above are referenced utilizing the same numerals. Still, it is noted that some features of the vestibular implant 400 will be different from that of the cochlear implant above. By way of example only and not by way of limitation, there may not be a microphone on the behind-the-ear device 126. Alternatively, sensors that have utilitarian value in the vestibular implant can be contained in the BTE device 126. By way of example only and not by way of limitation, motion sensors can be located in BTE device 126. There also may not be a sound processor in the BTE device. Conversely, other types of processors, such as those that process data obtained from the sensors, will be present in the BTE device 126. Power sources, such as a battery, will also be included in the BTE device 126. Consistent with the BTE device of the cochlear implant of FIG. 1, a transmitter/transceiver will be located in the BTE device or otherwise in signal communication therewith. Any one or more of the teachings herein can be used with the arrangement of FIG. 4.

The implantable component includes a receiver-stimulator in a manner concomitant with the above cochlear implant. Here, the vestibular stimulator comprises a main implantable component 120 and an elongate electrode assembly 14188 (where the elongate electrode assembly 14188 has some different features from the elongate electrode assembly 118 of the cochlear implant, some of which will be described shortly). In some embodiments, internal energy transfer assembly 132 and main implantable component 120 are hermetically sealed within a biocompatible housing. In some embodiments, main implantable component 120 includes a processing unit (not shown) to convert data obtained by sensors, which could be on board sensors implanted in the recipient, into data signals.

Main implantable component 120 further includes a stimulator unit (also not shown) which generates electrical stimulation signals based on the data signals. The electrical stimulation signals are delivered to the recipient via elongate electrode assembly 14188.

It is briefly noted that while the embodiment shown in FIG. 4 represents a partially implantable vestibular implant, embodiments can include a totally implantable vestibular implant, such as, where, for example, the motion sensors are located in the implantable portion, in a manner analogous to a cochlear implant.

Elongate electrode assembly 14188 has a proximal end connected to main implantable component 120, and extends through a hole in the mastoid 119, in a manner analogous to the elongate electrode assembly 118 of the cochlear implant, and includes a distal end that extends to the inner ear. In some embodiments, the distal portion of the electrode assembly 14188 includes a plurality of leads 410 that branch out away from the main body of the electrode assembly 118 to electrodes 420. Electrodes 420 can be placed at the base of the semicircular ducts as shown in FIG. 4. In an exemplary embodiment, one or more of these electrodes are placed in the vicinity of the vestibular nerve branches innervating the semicircular canals. In some embodiments, the electrodes are located external to the inner ear, while in other embodiments, the electrodes are inserted into the inner ear. Note also while this embodiment does not include an electrode array located in the cochlea, in other embodiments, one or more electrodes are located in the cochlea in a manner analogous to that of a cochlear implant.

Returning back to hearing prosthesis devices, and in particular a cochlear implant, FIG. 1E is a side view of the internal component (implantable component) of cochlear implant 100 without the other components of system 10 (e.g., the external components). The implantable portion of cochlear implant 100 comprises a receiver/stimulator 180 (combination of main implantable component 120 and internal energy transfer assembly 132) and a stimulating assembly or lead 118. Stimulating assembly 118 includes a helix region 182, a transition region 184, a proximal region 186, and an intra-cochlear region 188. Proximal region 186 and intra-cochlear region 188 form an electrode array assembly 190. In an exemplary embodiment, proximal region 186 is located in the middle-ear cavity of the recipient after implantation of the intra-cochlear region 188 into the cochlea. Thus, proximal region 186 corresponds to a middle-ear cavity sub-section of the electrode array assembly 190. Electrode array assembly 190, and in particular, intra-cochlear region 188 of electrode array assembly 190, supports a plurality of electrode contacts 148. These electrode contacts 148 are each connected to a respective conductive pathway, such as wires, PCB traces, etc. (not shown) which are connected through lead 118 to receiver/stimulator 180, through which respective stimulating electrical signals for each electrode contact 148 travel.

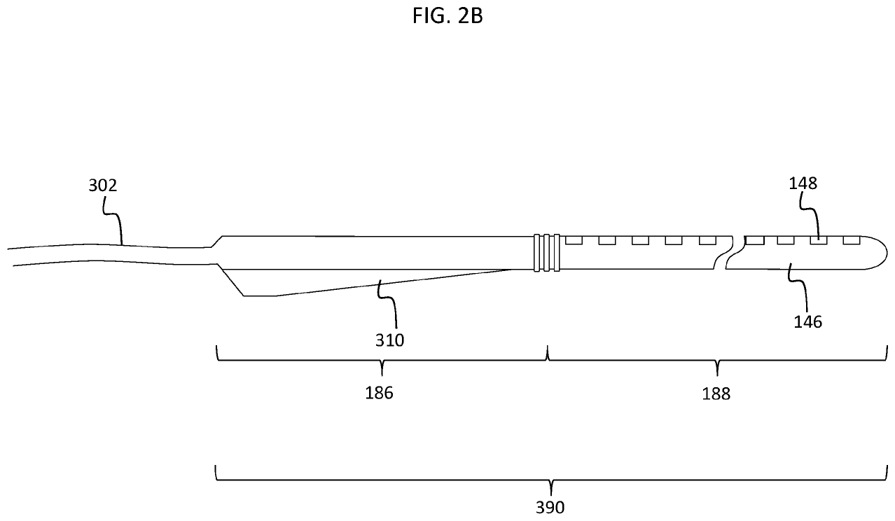

FIG. 2A is a side view of electrode array assembly 190 in a curled orientation, as it would be when inserted in a recipient's cochlea, with electrode contacts 148 located on the inside of the curve. FIG. 2A depicts the electrode array of FIG. 1B in situ in a patient's cochlea 140.

FIG. 2B depicts a side view of a device 390 corresponding to a cochlear implant electrode array assembly that can include some or all of the features of electrode array assembly 190 of FIG. 1E. More specifically, in an exemplary embodiment, stimulating assembly 118 includes electrode array assembly 390 instead of electrode array assembly 190 (i.e., 190 is replaced with 390).

Electrode array assembly 390 includes a cochlear implant electrode array componentry of the 190 assembly above. Note also element 310, which is a quasi-handle like device utilized with utilitarian value vis-à-vis inserting the 188 section into a cochlea. By way of example only and not by way of limitation, element 310, which is a silicone body that extends laterally away from the longitudinal axis of the electrode array assembly 390, and has a thickness that is less than that of the main body of the assembly (the portion through which the electrical leads that extend to the electrodes extend to the elongate lead assembly 302). The thickness combined with the material structure is sufficient so that the handle can be gripped at least by a tweezers or the like during implantation and by application of a force on to the tweezers, the force can be transferred into the electrode array assembly 390 so that section 188 can be inserted into the cochlea.

FIG. 2C presents additional details of an external component assembly 242, corresponding to external component 142 above. It is noted that in a modified form, this device can be used with the other prostheses herein (e.g., some such embodiments might not have the ear piece 250).

External assembly 242 typically comprises a sound transducer 220 for detecting sound, and for generating an electrical audio signal, typically an analog audio signal. In this illustrative arrangement, sound transducer 220 is a microphone. In alternative arrangements, sound transducer 220 can be any device now or later developed that can detect sound and generate electrical signals representative of such sound. An exemplary alternate location of sound transducer 220 will be detailed below. As will be detailed below, a sound transducer can also be located in an ear piece, which can utilize the “funneling” features of the pinna for more natural sound capture (more on this below).

External assembly 242 also comprises a signal processing unit, a power source (not shown), and an external transmitter unit. External transmitter unit 206 (sometimes herein referred to as a headpiece) comprises an external coil 208 and, a magnet (not shown) secured directly or indirectly to the external coil 208. The signal processing unit processes the output of microphone 220 that is positioned, in the depicted arrangement, by outer ear 201 of the recipient. The signal processing unit generates coded signals using a signal processing apparatus (sometimes referred to herein as a sound processing apparatus), which can be circuitry (often a chip) configured to process received signals-because element 230 contains this circuitry, the entire component 230 is often called a sound processing unit or a signal processing unit. These coded signals can be referred to herein as a stimulation data signals, which are provided to external transmitter unit 206 via a cable 247. In this exemplary arrangement of FIG. 1D, cable 247 includes connector jack 221 which is bayonet fitted into receptacle 219 of the signal processing unit 230 (an opening is present in the dorsal spine, which receives the bayonet connector, in which includes electrical contacts to place the external transmitter unit into signal communication with the signal processor 230). It is also noted that in alternative arrangements, the external transmitter unit is hardwired to the signal processor subassembly 230. That is, cable 247 is in signal communication via hardwiring, with the signal processor subassembly. (The device of course could be disassembled, but that is different than the arrangement shown in FIG. 1D that utilizes the bayonet connector.) Conversely, in some embodiments, there is no cable 247. Instead, there is a wireless transmitter and/or transceiver in the housing of component 230 and/or attached to the housing (e.g., a transmitter/transceiver can be attached to the receptacle 219) and the headpiece can include a receiver and/or transceiver, and can be in signal communication with the transmitter/transceiver of/associated with element 230.

FIG. 1F provides additional details of an exemplary in-the-ear (ITE) component 250. The overall component containing the signal processing unit is, in this illustration, constructed and arranged so that it can fit behind outer ear 201 in a BTE (behind-the-ear) configuration, but may also be worn on different parts of the recipient's body or clothing.

In some arrangements, the signal processor (also referred to as the sound processor) may produce electrical stimulations alone, without generation of any acoustic stimulation beyond those that naturally enter the ear. While in still further arrangements, two signal processors may be used. One signal processor is used for generating electrical stimulations in conjunction with a second speech processor used for producing acoustic stimulations.

As shown in FIG. 1F, an ITE component 250 is connected to the spine of the BTE (a general term used to describe the part to which the battery 270 attaches, which contains the signal (sound) processor and supports various components, such as the microphone—more on this below) through cable 252 (and thus connected to the sound processor/signal processor thereby). ITE component 250 includes a housing 256, which can be a molding shaped to the recipient. Inside ITE component 250 there is provided a sound transducer 220 that can be located on element 250 so that the natural wonders of the human ear can be utilized to funnel sound in a more natural manner to the sound transducer of the external component. In an exemplary arrangement, sound transducer 242 is in signal communication with remainder of the BTE unit via cable 252, as is schematically depicted in FIG. 1F via the sub cable extending from sound transducer 242 to cable 252. Shown in dashed lines are leads 21324 that extend from transducer 220 to cable 252. Not shown is an air vent that extends from the left side of the housing 256 to the right side of the housing (at or near the tip on the right side) to balance air pressure “behind” the housing 256 and the ambient atmosphere when the housing 256 is in an ear canal

Also, FIG. 2C shows a removable power component 270 (sometimes battery back, or battery for short) directly attached to the base of the body/spine 230 of the BTE device. As seen, the BTE device in some embodiments includes control buttons 274. The BTE device may have an indicator light 276 on the earhook to indicate operational status of signal processor. Examples of status indications include a flicker when receiving incoming sounds, low rate flashing when power source is low or high rate flashing for other problems.

In one arrangement, external coil 130 transmits electrical signals to the internal coil via an inductance communication link. The internal coil is typically a wire antenna coil comprised of at least one, or two or three or more turns of electrically insulated single-strand or multi-strand platinum or gold wire. The electrical insulation of the internal coil is provided by a flexible silicone molding (not shown). In use, internal receiver unit may be positioned in a recess of the temporal bone adjacent to outer ear 101 of the recipient.

With the above as a primer (the above should be considered base technologies from which we build upon, and are not part of the invention, but the teachings below can use any one or more of these features in some embodiments, providing that the art enables such), embodiments are directed to cochlear implants and other implants that, in some embodiments, utilize one or more of the teachings above, albeit modified in at least some instances, to practice the teachings herein.

FIG. 5 shows an implantable portion 500 of the cochlear implant, corresponding to portion 100 of FIG. 1A detailed above. In this regard, the features detailed above with respect to the cochlear implants are included in this embodiment. Element 181 corresponds to the RF antenna that receives transcutaneous magnetic inductive signals from the external component. The antenna 181 is in signal communication with electronics located in housing 185. Housing 185 is a hermetically sealed titanium housing that includes componentry of the cochlear implant that is configured to receive signals from the antenna 181 and, based on those signals, output signals to the electrodes of the electrode assembly 590, which can correspond to the electrode assembly 190 detailed above. Collectively, the electronics in the housing 185 and the antenna 181 establish a receiver-stimulator assembly 580, which can correspond to the receiver-stimulator assembly 180 detailed above. The antenna 181 and the housing 185 are located within a silicone body 183 that has been molded about those components. The electrode assembly 590 is in signal communication with the electronics of the housing 185 via lead assembly 589. The lead assembly can be a silicone body that is molded about electrical leads that run from the electrodes to a feedthrough that interfaces with the housing 185 to enable signal communication from the electronics in the housing to the electrical leads and thus to the electrodes. The electrode assembly, and in particular, the silicone body thereof, can be made separate from the silicone body that envelops the housing 185. In this regard, in an exemplary embodiment, the lead assembly 589 along with the electrode array assembly 590 is connected to the housing 185, or more accurately, the lead wires of the lead assembly 589 are first attached to a feedthrough that interfaces with the housing 185, thus placing the lead assembly into electrical signal communication with the receiver-stimulator assembly 580, which lead wires are supported by the silicone body that envelops the lead wires of the lead assembly 589. Then, silicone is molded about the housing 185 to form the silicone body 183, which silicone body traps or otherwise adheres the lead assembly 589 to the silicone body.

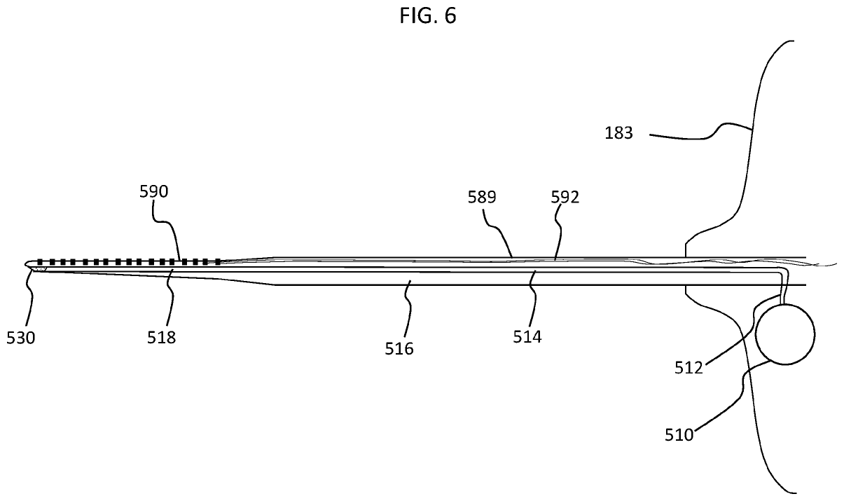

This embodiment further has a feature of a therapeutic substance delivery subsystem. In this regard, the embodiment of FIG. 5 is configured to deliver a therapeutic substance from outside the cochlea to inside the cochlea when the electrode array 590 is located in the cochlea. In this regard, the implantable portion includes a therapeutic substance delivery subsystem as just noted. This subsystem includes a cistern 510, as seen in FIG. 5, that forms part of a reservoir fill assembly (additional details of this will be described below). FIG. 6 shows additional details of the therapeutic substance delivery subsystem. In particular, the cistern 510 is shown in fluid communication with a conduit that includes a first portion 512 that leads to a second portion 514. Here, the cistern 512 is supported by the silicone body 183 that envelops the housing. In some embodiments, the cistern is completely subsumed within the silicone body 183, while in other embodiments, a portion of the cistern extends out of or otherwise is flush with a surface of the silicone body 183. The cistern 510 can be made out of titanium or a polymer that is stable with the therapeutic substance that will be placed into the cistern. The cistern 510 can include a septum at a top portion thereof, that can enable the cistern to be charged with a therapeutic substance. Some additional details of this will be describe below.

The conduit can be a polymer tube or be established by a polymer tube that is stable with the therapeutic substance. The tube interfaces with the cistern in any manner that can enable fluid to transfer from the cistern to the tube (an interference fit of a male outlet of the cistern over the tube can be used, for example). The tube runs from the cistern 510 into the silicone body 516 that envelops the electrical leads 592 that run from the electrodes. (Electrical leads could be located in the tube, while in other embodiments, the tube is “parallel” to and adjacent to the leads.) Note further that in an embodiment, the tube(s) could be located outside the silicone body 516 and run parallel with the silicone body (connected along the length, such as by straps or some connector that holds the tube(s) against the body along the length thereof) or loose so that the tube can be moved away from the silicone body, where the end of the tube then meets the electrode array (roughly analogous to how the old Chesapeake Bay Bridge Tunnel operated—bridges were separate from each other, but the bridges joined each other/met at the tunnels—here, the tube and the body could meet at the array, so that there is only one opening into the cochlea). In this regard, in an exemplary embodiment, the cistern 510 and the portion of the tube 512 that extends out of the silicone body 516 can be molded in the silicone body 183 during the molding process of the silicone body 183 about the housing 185. Portion 514 of the tube and at least some of the portion 512 of the tube can be placed with the electrical leads 592 and then silicone can be molded around both at the same time to establish the body 516. In an embodiment, there is no definitive structural tube per se, but instead a conduit within the silicone, that is a hollow space therein. For example, the hollow space can be a hollow space left over from removing a mandrel about which the lead is molded around. In an embodiment, silicone can be removed by hogging out a hollow space for example. Any device, system and/or method that can enable a passageway within the implant to be established that can enable the teachings herein can be used in some embodiments providing that the art enables the process and/or the end result. Thus, in an embodiment, the implant is “tubeless” vis-à-vis the therapeutic substance deliver system, in whole or in part (e.g., portion 514 and/or 518 could be tubeless, but portion 512 could be a tube for example. Any portion can be tubeless or be established by a tube.

Concomitant with the embodiments above, the electrode assembly 590 is located at the end of the lead assembly 589. The tube extends into the electrode array portion shown. The portion 518 of the tube that extends into the electrode array portion extends to the tip of the electrode array 590. Collectively, the portions 518, 514, 512 and the cistern form an implantable therapeutic substance reservoir. In an embodiment, portion 518 is configured to be flexible. In an embodiment, all portions or a majority of the portions of the therapeutic substance delivery system that are located intracochlearly when fully implanted are flexible. In an embodiment, the portions located intracochlearly are at least as flexible as the electrode array would be without the portion 518. Thus, in some embodiments, the portion 518 is even more flexible than the electrode array without the portion 518.

A plug 530 is located at the end of the tube (interference fitted inside the tube, for example, or bonded to the tube). The plug provides a bacterial seal at the tube portion 518 and thus the reservoir assembly, but enables the therapeutic substance in the reservoir to pass through, and thus into the cochlea. Additional details of this will be described below.

In view of the above, in an embodiment, there is an apparatus, such as an implantable portion of a cochlear implant, which includes an array of electrodes, and an implantable therapeutic substance reservoir. The apparatus is configured so that the therapeutic substance reservoir extends from a location behind an ear canal of a human between a mastoid bone and skin of the human to the cochlea when the apparatus is fully implanted in a recipient. In this regard, as noted above, the cistern, which is part of the reservoir, is adjacent the receiver-stimulator 580. When the implant is implanted in a head of a human, the cistern 510 is located behind and/or above the ear canal, or at least a portion thereof is so located. In an embodiment, at least 20, 25, 30, 35, 40, 45, 50, 55, 60, 65, 70, 75, 80, 85, 90, 95, or 100% of the overall area subsumed by the cistern (when looking down at the coil) is so located. Note further that in an embodiment, when looking directly at the side of the head (90 degrees from the front), with respect to a 50 percentile human factors engineering human of 60 years of age born in the United States, the implantable portion is configured so that any one or more of the aforementioned features vis-à-vis the cistern 510 is also applicable an area that is not overshadowed by the pinna of that human (for example 100% is not overshadowed, or at least 20% is not overshadowed). Note that this is not a subjective feature with respect to a given human. This is an objective feature that relates to a qualifiable fact relating to the aforementioned 50 percentile human. That said, in an exemplary embodiment, this can be a subjective value for a given person.

With respect to FIG. 8, there is a quadrant system presented that is centered about the ear canal 106 of the recipient. As can be seen, it is established by a vertical line 99 and a horizontal line 98 centered at the center of the ear canal 106. These lines establish four quadrants about the ear canal: Q1, Q2, Q3, and Q4. As will be understood, these quadrants generally follow the 12 hour clock, with quadrant 1 falling between the 12 o'clock position and the 3 o'clock position, quadrant 2 falling between the 3 o'clock position and the 6 o'clock position, quadrant 3 falling between the 6 o'clock position and the 9 o'clock position, and quadrant 4 falling between the 9 o'clock position and the 12 o'clock position. In an embodiment, the cistern falls completely within quadrant Q4. Accordingly, in an exemplary embodiment, there is a hearing prosthesis device that includes a reservoir that has a portion that falls within quadrant 4. The quadrants are established by the outermost opening of the ear canal 106, when looking directly at the side of the human (90 degrees from the front). These are established at the outermost portion where ear canal establishes a closed circle or oval, etc., in cross-section.

Note that there can be alternate quadrants. In an exemplary embodiment, quadrants can be established by lines 94/96, which are the topmost and back most tangent lines of the ear canal 106 (the opening of the ear canal), lines 99 and 98 as just detailed which correspond to the center of the opening of the ear canal), and lines 95 and 97 which correspond to the forward most and bottom most tangent lines of the ear canal (again, vis-à-vis the opening). The just noted features can be applicable to any of the quadrants established by any of these lines. (Quadrants can include a quadrant based on line 97 and line 94—all lines can be mixed and matched.)

Note further that in an exemplary embodiment, there can be quadrants that are based on lines parallel to any one or more of the lines shown in FIG. 8, wherein the lines are 0.5, 1, 1.5, 2, 2.5, 3, 3.5, 4, 4.5, 5, 5.5, or 6 cm or any value or range of values therebetween in 1 mm increments to the left or right or above or below a given line. Any of the aforementioned features can be applicable to such a quadrant.

In an embodiment, at least 30, 35, 40, 45, 50, 55, 60, 65, 70, 75, 80, 85, 90, 95%, or more, or any value or range of values therebetween in 1% increments (e.g., 33%, 54%, 31 to 94%, etc.) of the total volume of the reservoir can be located behind (where the face of the human is the front) and/or above (where the feet of the human are below) any one or more of the just noted lines.

In an embodiment, the reservoir is completely integrated in the implantable portion of the cochlear implant. For example, as detailed above, the cistern 510 is located within the silicone body that envelops the housing 185, and the tube portions 512, 514, and 518 and the plug 530 are all within the boundary that establishes the lead assembly with the electrode assembly.

In an exemplary embodiment, of the total outer surface area of the components that make up the reservoir (e.g., cistern and tubes and plug), at least 50, 55, 60, 65, 70, 75, 80, 85, 90, 95, or 100% of that surface area is in direct contact with silicone that establishes the silicone body that encases the housing and the silicone body of the lead assembly, including the electrode assembly. As will be detailed below, some portions of the cistern, such as the septum that enables the reservoir to be filled, can be located proud of the silicone body that encases the housing or otherwise flush with the surrounding body of silicone.

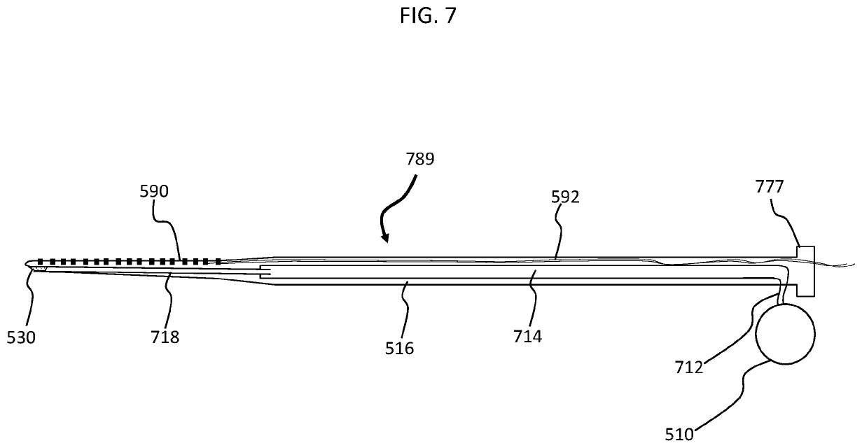

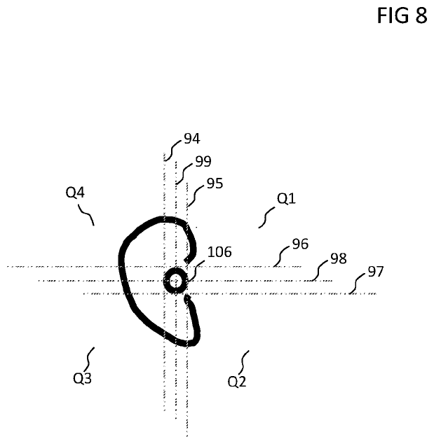

As noted above, there are assembly techniques associated with the teachings detailed herein, which assembly techniques give rise to structure that has utilitarian value with respect to implementing some of the teachings detailed herein. In this regard, FIG. 7 shows a lead assembly 789 prior to connection of that lead assembly to the receiver-stimulator of the cochlear implant. In an exemplary embodiment, this lead assembly is manufactured separately from the receiver-stimulator. As seen on the right end of the lead assembly 789, there is a T-shaped component 777. An exemplary embodiment, this is an extension of the silicone body 516. This can have utilitarian value with respect to establishing a portion of the lead assembly 789 that can establish a true interference fit when the silicone body 183 is placed around the housing. In this regard, the silicone body of the housing, during the molding process, will extend to the left of the T and around the T and thus around the cylindrical portion of the body 516. When the silicone solidifies, the T portion will be trapped within the silicone body. As can be seen in FIG. 7, lead assembly 789 includes the therapeutic substance reservoir. As can be seen, the cistern 510, and the associated tubes and plug are all part of the lead assembly. That is, when the lead assembly manufacture is completed, all of those parts will be present. Accordingly, when the lead assembly is attached to the receiver-stimulator, or more accurately, when the silicone is molded about the housing of the receiver-stimulator, the reservoir will become completely integrated in the implantable portion in one fell swoop. Put another way, during manufacture, the entire lead assembly of FIG. 7 is laid up against the receiver-stimulator, and then the silicone body is molded about the housing, and thus about the proximal portion of the lead assembly, and thus around at least portions of the cistern, to establish the integrated therapeutic substance delivery system, and thus the integrated reservoir.

It is noted that some embodiments do not utilize the T 777.

FIG. 7 also shows a non-mutually exclusive feature associated with the tubing of the lead assembly. In particular, two different tubes are utilized. Here, there is a first tube that has portions 714 and 712, which tube is a silicone tube. This tube can be easily bent or deformed so as to establish the portions 714 and 712 without permanently kinking the tube or otherwise creating a detrimental effect to the flow of the therapeutic substance therein. Lead 789 further includes tube 718, which is a polyimide tube. In this exemplary embodiment, tube 718 is interference fitted inside portion 714 as shown, but in other embodiments, the tubes can be bonded together (both a bond and an interference fit can be executed). This would not be a reservoir completely integrated with the lead assembly (prior to attachment to the receive-stimulator, more on this in a moment), in that the cistern is readily removable from the remainder of the lead assembly without damaging the reservoir (if for some reason the portion 712 was permanently fixed to the cistern, this would be different). And corollary to this is that portion 712 could be a separate component from portion 714 (portion 712 could be a first tube connected to tube 714, and could be disconnectable from tube 714 without destroying (e.g., cutting)). Indeed, portion 712 could be permanently fixed to the cistern 510, and portion 712 could be the way that the cistern is indirectly attached to the tube 714 (via portion 712).

In an embodiment, the carrier member 146 of the electrode array is molded about the tube 718 by itself or with tube 718 connected to tube 714. In an embodiment, the carrier member 146 of the electrode array is molded about portion 518. Subsequently, the silicone body 516 of the lead assembly is molded about tube 714 (and/or portion of tube 718 depending on the length of tube 718)/about portion 714. That said, in an embodiment, the carrier member is established with the silicone body 516 and thus those portions are monolithic with each other.

Thus, in an embodiment there is an electrode array that includes tube 718 and/or portion 518.

We now distinguish between the lead assembly and the lead portion of the implantable component. The lead assembly is the component that is attached to the housing and the receiver-stimulator during manufacturing, and can be identified after manufacturing, as would a bracket welded to a pressure vessel can be distinguished from the pressure vessel afterwards. Corollary to this is that the implantable component has a lead portion after manufacture of the implantable component or otherwise the completed implantable component that is obtained by doctors or surgeons or healthcare professionals for implantation (the surgeons do not attach the lead assembly to the receiver-stimulator; the components are delivered as one single apparatus in a completed form ready for implantation). The lead portion is the portion of the external component that extends from the feedthrough of housing 185 (the feedthrough to which the electrical leads are attached) to the most distal end of the electrode assembly 590, and this can take on portions of the silicone body that for example encases the electrical leads, and, in this embodiment, the cistern 510 and the portion of the tube 712 for example. In an exemplary embodiment, such as where the feedthrough is located on the left side of the housing 185 directly facing the lead assembly 589 (as opposed to being on the bottom or top or side of the housing), the lead portion of the implantable component would be the portion to the left of reference line 599. This would thus include a portion of the silicone body 183 that encases the housing 185 (which would also encase portions of the electrical leads for example) if the leads extended from the silicone body of the lead assembly prior to the establishment of the silicone body 183. And there are some embodiments where the feedthrough is located on the bottom or the top of the housing 185 or on the sides of the housing (side relative to the side facing the electrode array 590 in the arrangement of FIG. 5). Thus, the lead portion of the implantable component could extend to the right of the reference line 599 (as opposed to ending there) but in a potentially narrow channel (which may or may not be distinct) encompassing the wire leads of the lead assembly 589. Indeed, in an embodiment, there can be a channel in the bottom of the housing 185 that provides room for the electrical leads. The lead portion would be the electrical leads in that channel and the portion of the silicone body that fills at least a portion of the channel to secure the lead relative to the housing 185 between the feedthrough and the silicone body of the lead assembly 589. Thus, when looking on the side, there could have a dog leg sub portion that extends downward from the front of the housing 185 (from the left side of reference 599) and then underneath the housing 185. Note that in these embodiments, the lead portion would include everything to the left of reference 599.

Embodiments thus include a reservoir assembly that is completely located, relative to the embodiment of FIG. 5, to the left of line 599, which line represents the leftmost portion of the housing 185. In an exemplary embodiment, with respect to the total interior volume of the reservoir, at least 90, 91, 92, 93, 94, 95, 96, 97, 98, 99, or 100% of that volume or any value or range of values therebetween in 1% increments is located to the left of line 599, and thus to the left of the leftmost portion of the housing 185.

By leftmost portion of the housing 185, with reference to FIG. 5, this is the portion that extends in the direction of the longitudinal axis of the implantable portion (the horizontal of FIG. 5) opposite the antenna 181.

By rough analogy, the portions of the silicone body 185 at issue that are part of the lead portion can be considered akin to the mounting portion of an antenna. The base of the antenna that includes a fixture so that the antenna can be attached to say the fuselage of an aircraft for example, would still be considered to be an antenna portion for example.

And it is again noted that portions of the silicone body 183 can overlap with the silicone body of the lead assembly 589.

Thus, we see embodiments where the reservoir is completely integrated into a lead portion of the cochlear implant, the lead portion including the array of electrodes.

In an embodiment, the reservoir has a volume of less than, greater than and/or equal to (all-inclusive or non-inclusive) 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 8, 9, 10, 11, 12, 13, 14, 15, 16, 17,1 8, 19, 20, 21, 22, 23, 24, 25, 26, 27, 28, 29, 30, 35, 40, 45, 50, 55, or 60 microliters, or any value or range of values therebetween in 0.01 microliter increments (e.g., 9.03, 22.22, 5.01 to 19.31 microliters, etc.) Thus, in an embodiment, the reservoir has a volume of no more than any one of those values (e.g., no more than 20 microliters, no more than 10 microliters, no more than 0.73 microliters, etc.). By “no more than,” that includes a volume that is less. The specification is that the volume does not exceed that amount. And the volume corresponds to the volume that can receive therapeutic substance.

In an embodiment, the cistern has an interior volume (fluid capacity) that is less than, greater than and/or equal to 2, 3, 4, 5, 6, 7, 8, 9, 10, 11, 12 microliters or any value or range of values therebetween in 0.005 microliter increments. In an embodiment, the tube 714 (alone or including portion 712) has an interior volume (fluid capacity) of 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6. 3.7, 3.8, 3.9, 4, 4.1, 4.2, 4.3, 4.4, 4.5, 4.6, 4.7, 4.8, 4.9, or 5 microliters or any value or range of values therebetween in 0.005 microliter increments. In an embodiment, the tube 718 has an interior volume (fluid capacity) of 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, or 0.3 microliters, or any value or range of values therebetween in 0.001 microliter increments. In an embodiment, the length of tube 718 is less than greater than and/or equal to 6, 7, 8, 9, 10, 11, 12, 13, 14, 15, 16, 17, 18, 19, 20, 21, 22, 23, 24, 25, 26, 27, or 28 mm, or any value or range of values therebetween in 0.1 mm increments. In an embodiment, the length of tube 714 (alone or including portion 712) is less than greater than and/or equal to 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or 48 mm, or any value or range of values therebetween in 0.1 mm increments. The outer diameter of the cistern can be less than, greater than and/or equal to 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, or 3.5 mm, or any value or range of values therebetween in 0.05 mm increments. In an embodiment, the inner diameter of tube 718 is less than, greater than and/or equal to 0.05, 0.06, 0.07, 0.08, 0.09, 0.1, 0.11, 0.12, 0.13, 0.14, 0.15, 0.16, 0.17, 0.18, 0.19, or 0.2 mm, or any value or range of values therebetween in 0.001 mm increments. In an embodiment, the inner diameter of tube 714 and/or portion 712 is less than, greater than and/or equal to 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45 mm, or any value or range of values therebetween in 0.001 mm increments.

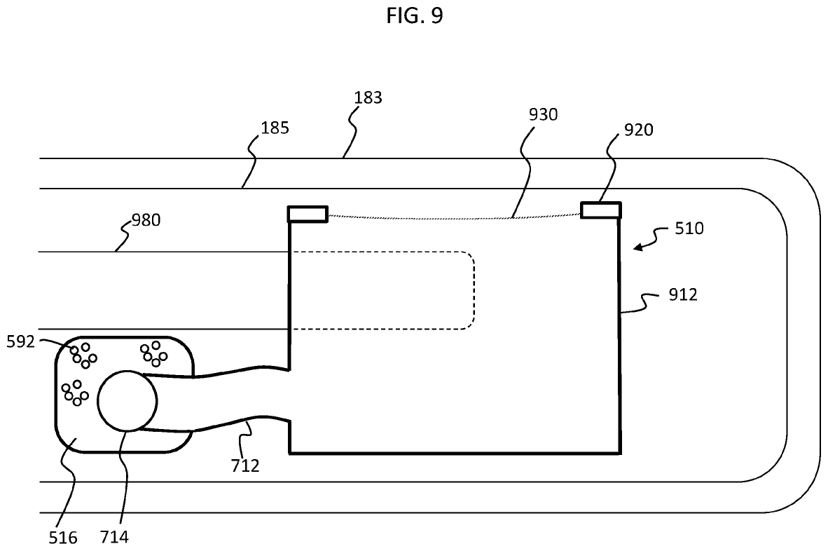

An embodiment is such that the apparatus includes an integrated septum configured to receive a termination of a syringe to enable a therapeutic substance to be delivered to the reservoir, which integrated septum provides a bacterial seal between the reservoir and an outside environment of the apparatus after the termination is removed. FIG. 9 shows an exemplary embodiment of such, which is a view with respect to section 9-9 of FIG. 5. Here, additional details of an exemplary cistern according to an exemplary embodiment are described. The cistern 510 includes a titanium housing wall 912 to which is attached support fixture 920 that is a titanium ring that is welded to the top of the cylindrical wall 912 of the cistern. In an embodiment, the wall or other portions of the housing can be made of PEEK or any other engineering polymer that can have utilitarian value or otherwise enable the teachings herein, or can be made of stainless steel, or CoCr, or could also be ceramic or 3D printed Ti, with a glass/ceramic/epoxy/polymer lining.

The support fixture holds a septum 930. The material of the septum is clamped in between portions of the titanium ring 920 or otherwise bonded to the interior side walls of the ring 920.

Embodiments can include the utilization of a self-healing septum. The septum 930 is configured to permit at least one puncturing (and in some embodiments repeated puncturing) and subsequent healing by a termination of a syringe. The termination can be inserted through the septum so that a therapeutic substance can be injected into the cistern, and thus “charge” or otherwise convey the substance into the reservoir. That said, in an embodiment, the septum can be a one time use/single use septum. And in this regard, in an exemplary embodiment, the reservoir or otherwise a therapeutic substance delivery system is a one time use system. In an embodiment, it is utilized for some chronic diseases or otherwise some chronic ailments. In an exemplary embodiment, the design is specifically such that after implantation, the therapeutic substance delivery system or otherwise the reservoir cannot be recharged with therapeutic substance without executing a more than minimally invasive procedure (in an exemplary embodiment, a minimally invasive procedure would be utilizing a termination of a syringe to pierce the skin to reach the implantable portion). In an exemplary embodiment, the only way to recharge the therapeutic substance delivery system or otherwise the reservoir would be to cut into the skin making an incision having a length that is at least ¼ of an inch in length. In an exemplary embodiment, the only way to recharge a therapeutic substance delivery system is to actually lift at least a portion of the implantable component away from the skull or otherwise out of the pocket within the human to access such and otherwise manipulate such. In an embodiment, the device has to be physically altered to recharge the device. In an embodiment, the device simply cannot be recharged after implantation.

And while embodiments above have focused on the utilization of a charging device or a fill device or otherwise filling the reservoir just before implantation, in another embodiment, the therapeutic substance can be located in the reservoir while the therapeutic substance is located in the packaging and otherwise sterilely sealed in the packaging. In an embodiment, the packaging has an expiration date related to the therapeutic substance, which expiration date is located on the packaging or otherwise provided with the packaging. In an exemplary embodiment, the implantable component should be implanted by that expiration date or at a time related to that expiration date. This can also be the case with respect to the therapeutic substance that is provided in the fill device, but not provided in the reservoir until the package is opened or just before the package is opened. In an embodiment, the reservoir can be configured to be “drained” to enable the therapeutic substance therein to be removed, and replaced with different substance, in form or type or simply new amounts of the same therapeutic substance. In this way, if the therapeutic substance is expired, even if in the reservoir, the implantable component can be used.

Indeed, embodiments include selecting a specific type of therapeutic substance to be delivered by the implantable portion, and then charging the implantable portion within the affirmation time periods herein. Note further that embodiments can include packaging two or more different types of therapeutic substances instead of just one single therapeutic substance. In this exemplary embodiment, this can enable the healthcare professional to choose which therapeutic substance to utilize just prior to implantation. Further, a plurality of the same therapeutic substance can be provided at different strengths to allow the healthcare provider to choose the strength to be delivered. Corollary to this is that in some embodiments, the therapeutic substance can be such that the substance itself determines the diffusion rate. For example, the same therapeutic substance could be provided in a mixture that diffuses more slowly relative to that same therapeutic substance provided in another mixture. All of this can be chosen just prior to implantation.

And note that embodiments include obtaining an implantable portion in accordance with the teachings detailed herein, but not charging or otherwise placing therapeutic substance therein. That is, embodiments can include the reservoirs, but do not always use those reservoirs to deliver therapeutic substance. In an exemplary embodiment, this could be because there is no reason to with respect to a particular demographic and/or the particular background and/or particular aetiology of the recipient.

In an embodiment, the therapeutic substance delivery system can be “disabled” or otherwise adapted in a scenario where there will be no therapeutic substance utilized in the device. In an exemplary embodiment, the distinct barriers could be treated with a substance that eliminates the porosity and/or creates a barrier to diffusion. Indeed, in an exemplary embodiment, instead of providing a therapeutic substance to the reservoir, a biocompatible material, such as silicone, could be injected into the reservoir. By rough analogy, this can be the equivalent of filling completely empty old heating oil tanks (that are clean inside) that are in the ground with concrete. In an embodiment, another way to switch off the delivery of substance is to replace the drug solution with an artificial perilymph or saline for injection/delivery into the cochlea.

In an exemplary embodiment, the therapeutic substances located in the packaging are stable for at least one year from the date that the substances are sealed within the packaging.

Embodiments have focused on shipping the therapeutic substance in the same package with the implantable portion. Embodiments include shipping two separate packages or otherwise having two separate packages, one for the therapeutic substance and the other for the implantable portion. This way, if the implantable portion is not used within a certain period of time beyond the expiration date, all that needs to be happening is that the therapeutic substance can be disposed of. Also, this can enable the therapeutic substance to be used earlier than that which is the case.

Embodiments also include stocking different types of implantable portions. Some of them have the therapeutic substance delivery systems detailed above, while others do not. In an exemplary embodiment, depending on the needs of the recipient, the package with the therapeutic substance delivery system, or more accurately, the package with the implantable portion that includes the therapeutic substance delivery system will be opened if needed, and if not, the packages with the implantable portion that do not include the therapeutic substance delivery system will be utilized. In an exemplary embodiment, for example, half, or ⅓, or ¼, or something along those lines of the implantable portions and storage would have the therapeutic substance delivery system, and the remainder would not. In this manner, depending on the needs of the recipient, a given device would be selected. Corollary to this is that in some embodiments, therapeutic substance is separately package, and thus there might be a number of doses that equal to half or ⅓, etc. of all of the implantable portions that are stored at a healthcare facility. This because not all implantable portions would utilize therapeutic substance or otherwise be charged with such.

Still, embodiments include shipping together the therapeutic substance and the implantable portion, whether in the same packaging or in separate packages but shipped together.

Embodiments can enable repeated sealingly access from outside the recipient via the septum. This can be achieved, by way of example, by extending a termination of a syringe through skin over the septum and then puncturing the septum, thus enabling the reservoir to be “refilled’ or recharged after implantation.

The septum seals the top of the cistern and otherwise establishes a barrier between the reservoir and the tissue above the mastoid bone/the internal portion of the human proximate the proximal portion of the lead portion of the implantable component. In an exemplary embodiment, septum 930 is configured to receive and otherwise permit a termination of a syringe, such as that of a hypodermic syringe, to pass therethrough in a manner analogous to or otherwise the same as liquid medical containers that include septums (self-healing septums) that enables the termination of the syringe to pass therethrough to access the liquid therapeutic substance in the container. In at least some exemplary embodiments, any device, system, and/or method that will enable repeated sealingly access from outside the external component (whether implanted in some embodiments or not implanted in others (and the two need not be mutually exclusive, but can be so)—as will be described below, the septum can be used prior to implantation to enable the reservoir to be charged) can be utilized in some embodiments.

In some embodiments, a cap can be placed over the septum to isolate the septum from the environment. This can be utilitarian, for example, in a scenario where it is intended to only charge the reservoir once. Thus, in an embodiment, prior to implantation, a surgeon or other healthcare professional can charge the reservoir, by extending a termination of a syringe through the septum, and then after withdrawing the termination, placing a titanium disc or some other rigid body over the lumen, to provide further protection. In an exemplary embodiment, this component that is placed over the lumen can further seal the reservoir at the top of the cistern, providing a level of redundancy vis-à-vis sealing the reservoir (and otherwise providing protection to the septum from shock or some feature change resulting from long term exposure to body fluids that would otherwise interact with the septum in the absence of the cap).

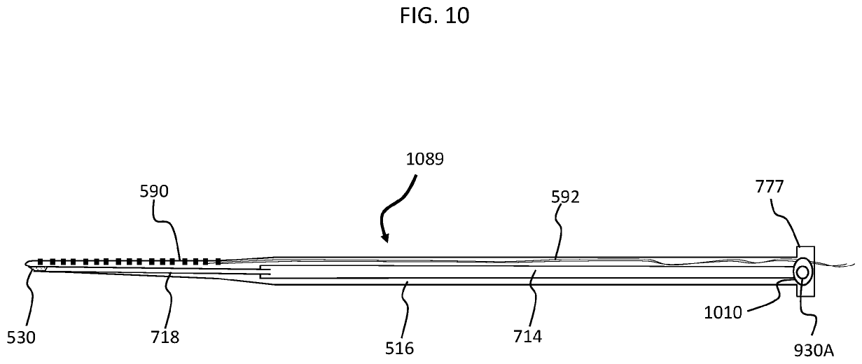

Concomitant with the embodiment above where the cistern is part of the lead portion of the implantable component, the septum can also be part of the lead assembly of the implantable component. Embodiments can include changing/having a different lead assembly cross-section, at least at/proximate the cistern, to make charging/filling the cistern less cumbersome. For example, a rectangular cross-section or a cross-section with a flat bottom can be used so that the lead does not “jump around” when a termination or the like is passed into the cistern to charge/fill the cistern, such as might be the case with a round/circular cross-section lead assembly (the lead assembly could roll to one side when the force of the termination is applied to the cistern/septum. And in this regard, depending on the size of the cistern (if a cistern is even desired-some embodiments just have a tube 714), the cistern and thus the entire reservoir could be completely integrated into the lead assembly. FIG. 10 presents an exemplary embodiment, which includes a self-healing septum 930A, which is located in the T portion 777 of lead assembly 1089. In this regard, the larger volume of the T portion of the silicone body 516 is utilized to enable access to the completely integrated reservoir. In this exemplary embodiment, the septum 930A is smaller than the septum 510 of the embodiment of FIG. 5 detailed above, owing to the smaller cistern 1010 size, but it is noted that the septum 930A could be larger than that shown-the septum could extend over a substantially large portion of the top of the cistern 1010, as is the case in the embodiment of FIG. 9.

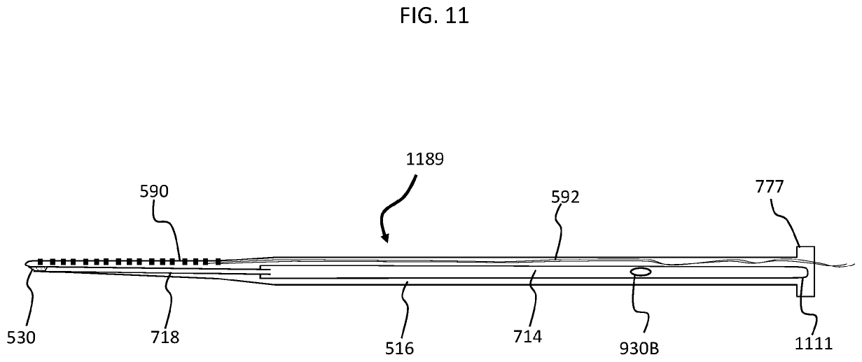

And with reference to embodiments that do not include a dedicated cistern, in an exemplary embodiment, the septum could be located on tube 714 for example, or on/in an adapter therefore. Indeed, tube 714 could have the septum feature on the outer surface thereof, such as the outer surface facing the viewer of FIG. 11 as shown. Here, there is a lead assembly 1189 that includes an oval shaped septum 930B located on the outer cylindrical surface of tube 714, where the proximal end 1111 of tube 714 is sealed (e.g., with a titanium plug or with a silicone plug or by joining the wall of the tube together). As seen in this embodiment, the septum 930B is located away from the silicone body 183 (or would be when the lead assembly 1189 is attached to the receiver-stimulator). Thus, to charge or otherwise fill the reservoir, the termination of the syringe that is utilized would only pierce the silicone body 516 of the lead assembly. And note that in some embodiments, the silicone body does not extend over the septum such as can be the case with the embodiments where the cistern is located elsewhere, such as the embodiment of FIG. 5. In some embodiments, all portions of the reservoir and/or cistern are covered by silicone, while in other embodiments, there is a portion that is not covered by silicone, such as the portion where the septum is located. And in this regard, in at least some exemplary embodiments, there is the action of piercing the silicone body with the termination of the syringe and then piercing the septum to charge the reservoir.

In view of the above, in an exemplary embodiment, there is an apparatus, comprising an implantable therapeutic substance reservoir, a plurality of electrodes (e.g., electrodes 148), a silicone carrier body (e.g., carrier 146) supporting the plurality of electrodes. Further, the apparatus includes a stimulator assembly including a housing and stimulation electronics. In this embodiment, the apparatus can include a receiver-stimulator such as receiver-stimulator 580 noted above, or only a stimulator, or some other componentry in addition to a stimulator. The point is this apparatus requires only a stimulator. And as noted above, embodiments can be applicable to pacemakers or the like where for example, there may not be a receiver component thereof. In this regard, any disclosure related to a receiver-stimulator corresponds to a disclosure of an alternate embodiment of a stimulator and/or a receiver unless otherwise noted providing that the art enables such. Put another way, any reference to a receiver-stimulator includes an alternate embodiment where there is only a receiver or only a stimulator, or a stimulator and some other components or additional components plural but no receiver. Corollary to this is that a receiver-stimulator or a receiver can include an assembly that has additional functionality beyond a receiver-stimulator, providing that there is functionality of the receiver-stimulator.

In this embodiment, the apparatus includes a fill port (e.g., the septum) in fluid communication with the reservoir configured to enable therapeutic substance delivery to the reservoir (e.g., such as by a termination of a syringe piercing the septum), and the fill port is between the housing and the plurality of electrodes. In an embodiment, the “between” is based on relative position relative to a longitudinal axis of the apparatus (in FIG. 5, the longitudinal axis is horizontal to the view thereof).

In an embodiment, the fill port is part of the lead portion as noted above. But note that in other embodiments, the fill port can be part of the stimulator portion (e.g., a portion to the right of line 599.) That is, some embodiments are different than the aforementioned embodiment just detailed. An exemplary embodiment, tube 714 extends around the housing 580 to a location inside the coil 181, where the fill port and/or a cistern is located. In an exemplary embodiment, the fill port is located to the side of the housing (above or below the housing with respect to the orientation FIG. 5).

In an exemplary embodiment, the fill port and/or the cistern is located no more than 1, 0.9, 0.8, 0.7, 0.6, 0.5, 0.45, 0.4, 0.35, 0.3, 0.25, 0.2, 0.15, or 0.1 cm, or any value or range of values therebetween in 0.01 cm increments away from the housing (that is at least one part is within those ranges). In an exemplary embodiment, the entire cistern and/or fill port is located within those ranges from the housing, or within an additional 0.5 cm as applied to any of those ranges. In an embodiment, the entire fill port and/or cistern is located between the housing 185 and the electrodes vis-à-vis the orientations above. In an exemplary embodiment, the aforementioned values are also applicable to this spatial relationship between the cistern and/or fill port and at least one or a plurality or all of the electrical lead wires of the lead assembly. In an exemplary embodiment, the aforementioned values are also applicable to the spatial relationship between the cistern and/or fill port and at least a portion or the entire feedthrough assembly (980 in FIG. 9) of the housing to which the electrical lead wires the lead assembly are attached. And thus, the fill port and/or the cistern can be between the feedthrough and the electrodes.

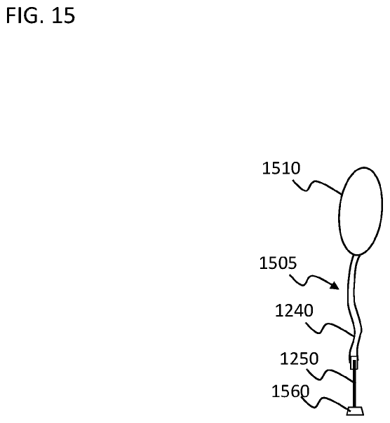

It is noted that in an alternate embodiment, the device of FIG. 15 can be located in the packaging, and in fluid communication with the implantable device. The termination 1250 could be sealed with a frangible component or a blister or the like. A professional can squeeze the component 1510 while it is sterilely sealed in the packaging to charge the therapeutic substance before opening the package. The pressure build up in the device of FIG. 15 can break the frangible seal or the blister. Thus the substance in the device of FIG. 15 is kept separate/isolated from the implant/maintained in the device of FIG. 15 unit right before surgery for example.

In an exemplary embodiment, at least 60, 65, 70, 75, 80, 85, 90, 91, 92, 93, 94, or 95% or, any value or range of values therebetween in 1% increments of the total internal volume of the reservoir (the volume that receives the therapeutic substance) is located no more than 1.5, 1.4, 1.3, 1.2, 1.1, 1, 0.9, 0.8, 0.7, 0.6, or 0.5 cm from reference line 599 or otherwise from the leftmost portion of the housing 185 and/or the interior volume of the reservoir according to any one or more of the percentages detailed above is located to the left of reference 599.

In an exemplary embodiment, the cistern is located above or below the electrical lead wires with respect to the frame of reference of FIG. 5. In this regard, while some embodiments depict the cistern located one side or the other side of the wires, in other embodiments the cistern and the wires overlap with respect to the frame of reference of FIG. 5. Note further that in some embodiments, two or more cisterns are present, one on either side of the wires. Note also that while the embodiments depicted in the figures show a cistern with a circular top arrangement, in an embodiment, the cistern can be more rectangular. FIG. 5A shows an exemplary embodiment of such, where cistern 510A extends from one side of the lead wires (not shown but compare to FIG. 6) over the lead wires to the other side of the lead wires. In an embodiment, the cistern 510A could be arched shape to extend over (or under) the electrical lead wires.

Concomitant with the teachings of the septum above, the fill port includes a resealable septum configured to receive a termination of a syringe, wherein the septum is configured provide a barrier to bacteria upon removal of the syringe from the septum after delivery of the therapeutic substance to the reservoir.

In an embodiment, the resealable septum is above or under electrode lead wires extending from the receiver-stimulator to the plurality of electrodes. In this regard, FIG. 9 shows the septum 930 above all of the electrical lead wires 592. Thus, an embodiment includes the resealable septum being above all of the electrical lead wires extending from the receiver-stimulator to the plurality of electrodes. (Above and below is with respect to a direction away from the bone facing side or towards a skin facing side of the implant. In FIG. 9, the skin facing side is the top and the bone facing side of the bottom.) In an embodiment, all of the septum is above all of the electrode wires. But as noted above, embodiments can include a septum that is below the electrical lead wires. Here, the cistern can be inverted relative to that shown in the figures. Or alternatively, one can consider the figures showing the bottom portion of the implantable component. In this regard, embodiments can include an arrangement where the cistern is filled only before implantation, and then never filled or otherwise recharged again. Thus, there is no “need” to have the septum facing the skin so that the septum can be pierced after implantation so that the reservoir can be refilled or otherwise recharged. In an embodiment, at least a portion of the septum is below some or all of the electrical lead wires. In an embodiment, all of the septum is below some or all of the electrical lead wires. And note by septum, it is not meant the septum retaining ring or the like. This references the actual septum.

Further, as can be seen, in an exemplary embodiment, a portion of the cistern is located above some or all of the electrical lead wires. In an embodiment, a portion of the cistern is located below some or all of the electrical lead wires. In an embodiment, there are one or more lead wires below the lowest portion of the cistern (all can be such), and in an embodiment, there are one or more lead wires above the topmost portion of the system (again, all of lead wires can meet this feature). In an embodiment, the aforementioned spatial values relating to the septum and/or fill portion and/or the cistern can be instead in relation to the feedthrough 980. That is, some or all of the fill port can be above or below some or all of the feedthrough.

Embodiments have focused on a cistern that is completely integral with the implantable component of the cochlear implant. Embodiments include a cistern that is not completely integral with the implantable component, one that is not meant to be implanted with the implantable component of the cochlear implant. Utilitarian value of this will be described in greater detail below, but briefly, an exemplary embodiment can enable the reservoir to be charged with therapeutic substance just before implantation of the implantable portion, or more accurately, more easily charged with therapeutic substance just before implantation of the implantable portion, and then the cistern of this embodiment can be removed from fluid communication with the reservoir.

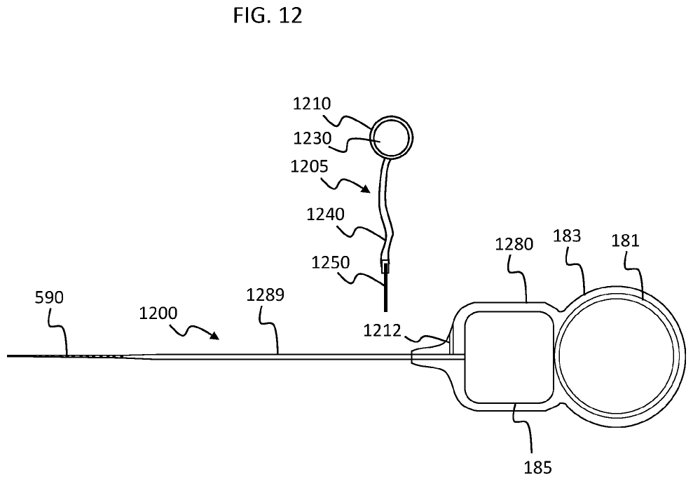

FIG. 12 presents an exemplary implantable portion 1200 along with a reservoir fill assembly 1205 that is removed from the implantable portion 1200. The reservoir fill assembly includes a cistern 1210 that has a septum 1230, which may or may not be self-healing owing to the fact that this arrangement is not implantable, and otherwise not to be implanted, where element 1230 can be any other type of arrangement that can enable the functionality thereof, which is to allow a termination of a syringe to pass therethrough into the interior volume of the cistern so that therapeutic substance can be transferred therein. A tube 1240, which can be a silicone tube or any of the tubes detailed above or any other tube that can have utilitarian value and otherwise can enable the teachings detailed herein, especially in view of the fact that assembly 1205 is not implanted, extends from the cistern 1210 in a manner analogous to how the tube extends from the implantable cistern detailed above. At the end of tube 1240 there is a termination 1250 in fluid communication with the interior of the tube 1240, and thus in fluid communication with the volume of the cistern 1230. The termination 1250 has a meeting component that mates with the tube 1240 and otherwise forms a fluid tight seal. Holding these thoughts for a moment, we now refer to the implantable portion 1200. The implantable portion 1200 includes a lead assembly 1289 that corresponds to any of the lead assembly's detailed above, but includes an opening in the tube 714 at the base of passageway 1212 that extends through a portion of the silicone body 183 that encases housing 185. In this regard, there is no portion 712 of the tube of the lead assembly 1289.

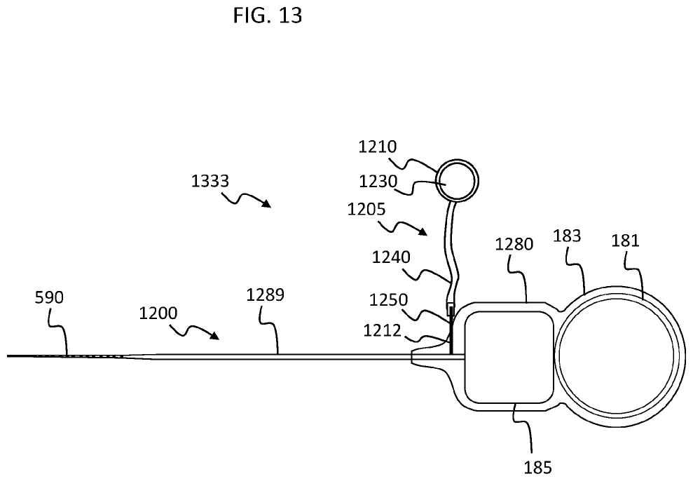

In this regard, there is no portion 712 of the tube of the lead assembly 1289. Instead, portion 712 has an opening (more on this below) that is sized and dimensioned to establish an interference fit with the termination 1250 of the fill assembly 1205. And to this end, FIG. 13 shows the fill assembly 1205 interfacing with the implantable portion 1200. Here, the termination 1250 has been pushed into the passageway 1212 in the silicone body 183 to reach the opening/orifice in the tube of the lead assembly 1289. The termination 1250 establishes an interference fit in the opening. When the cistern 1210 is charged with therapeutic substance from the syringe for example, the therapeutic substance flows from the cistern through tube 1240 through termination 1250 and into the tube of the lead assembly 1289. In an embodiment, this fills or substantially fills the tube in the lead assembly with the therapeutic substance. Alternatively, however much therapeutic substance is desired to be located in the tube is delivered utilizing the fill assembly 1205. Upon completion of filling or otherwise the transfer of the desired amount, the fill assembly 1205 is removed by pulling the termination 1250 out of the passageway 1212. In an embodiment, the passageway 1212 is a slit or otherwise a puncture, a pre-existing puncture or slit, of the silicone body 183. That is, the passageway 1212 does not result from coring of the body 183. Thus, the passageway 1212 is configured to collapse upon itself when the termination 1250 is removed from the passageway 1212. That is, the elastic nature of the silicone body 183 “closes” the passageway upon the removal of the termination 1250. Corollary to this is that the elastic nature of the silicone body 183 allows the passageway to open when the termination 1250 is pushed in the direction of the passageway 1212 from outside the body 183.

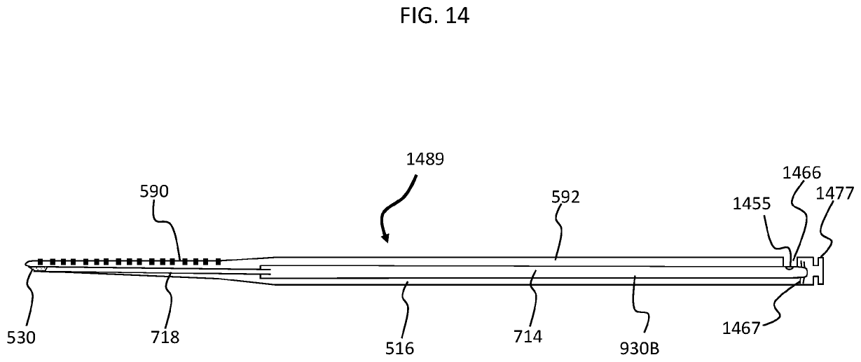

FIG. 14 shows an exemplary lead assembly 1489 that includes the orifice/opening 1455 detailed above. In this exemplary embodiment, the opening 1455 in tube 714 is undersized relative to the outer diameter of the termination 1250. Because tube 714 is silicone based, the tube 714 will expand to accept the termination 1250. Any other elastomeric tube can be utilized. That said, in some embodiments, the opening 1455 might be sized to establish an interference fit or slip fit or even an oversize fit vis-à-vis the termination 1250. Note further that while the embodiment presented in FIG. 14 presents an opening 1455 in the tube 714, in an alternate embodiment, a reinforcing grommet or an adapter can be located at the portion of the tube 714. Indeed, the end of the tube 714 could be affixed to an adapter or the like made of titanium or a rigid polymer or some other more rigid material than silicone, and could have an O-ring at the opening of the adapter to provide at least a modicum of fluidic sealing between the termination 1250 and the adapter. The adapter could “cap” the end of the tube at the distal end, and provide an opening at a 90° angle to the interface of the adapter with the tube, which opening would be sized and dimensioned to receive the termination 1250. Again, this opening could have an elastomeric O-ring or the like for sealing. In an embodiment, this adapter could have a one-way valve or the like to prevent the therapeutic substance from flowing out of tube 714 when the termination is removed after tube 714 is charged. That said, in an embodiment, it is the tube 714 that seals itself owing to the elastomeric nature of the material and the dimensioning of the opening 1455.

The above said, in an embodiment, it is the silicone body 183 that provides sealing (both can provide sealing for purposes of redundancy in some embodiments—a valve and/or construction of the opening 1455). As noted above, passageway 1212 is not a cored passage, but instead is simply established by the separation of material of the body 185. For example, a solid needle can be pushed from outside the body 183 to the tube 714 (or the adapter). A non-coring needle can be used. The solid needle simply separates the silicone. A round needle could be used, or a cutting device that has a height that is higher than a width (so the passageway is not “circular” when the cutting device is in the body, but is instead linear/rectangular, and thus a slit). That said, in some other embodiments, the material is actually removed from the passageway providing that the art enables such. Any arrangement of establishing the passageway and resulting passageway that can enable the teachings detailed herein can be utilized in at least some exemplary embodiments.

In an exemplary embodiment, upon removal of the termination 1250 from the passageway 1212, in at least some exemplary embodiments, the passageway 1212 collapses upon itself and provides a seal between the outside of the body 183 and the tube and/or adapter, thus preventing and/or at least limiting fluid transfer (transfer of the therapeutic substance) from the tube 1214 to outside the body 183 through the passageway 1212, sealing the reservoir.

Briefly referring back to FIG. 14, as can be seen, the lead assembly 1489 (where the electrical wires have been removed for clarity) shows a variation of an embodiment that increases the retention of the lead assembly to the body 183. Here, there is a T 1477, but the T is smaller and within the outer diameter or otherwise the maximum outer diameter of the silicone body 516. This as distinguished from the T configuration above, which extends beyond the outer boundaries of the body 516 in the lateral direction. Also shown is an opening 1466 in the body 516. This opening 1456 leads to the opening 1455 and tube 714. The opening 1456 is an absence of silicone of the body 516, thus providing clearance between the opening 1455 and the ambient environment prior to attachment of the lead assembly 1489 to the remainder of the implantable component. In an exemplary embodiment, when the body 183 is formed about the housing, and thus about the distal end of the lead assembly 1489, the silicone that establishes body 183 flows into the opening 1456. This can provide additional adherence between the body 183 and lead assembly 1489, but also provides for a uniform silicone body between the opening 1455 and the outside of the implantable component. This could have utilitarian value with respect to ensuring a straight path from outside the body 183 to the opening 1455. This as opposed to a scenario that might exist where there are two separate bodies; one of which is the body 516 of the lead assembly, and the other of which is the body 183 that encompasses the housing 185. The silicone of the two separate components may not necessarily bond perfectly or otherwise sufficiently to ensure that the portion of the passageway 1212 that is established through the body 516 of the lead assembly remains aligned with a portion of the passageway 1212 that is established through the silicone body 183.

In view of the above, it can be seen that in an embodiment, there is an assembly, such as assembly 1333 of FIG. 13, that includes an apparatus 1200 or any of the other implantable component(s) detailed herein and a reservoir fill assembly, such as assembly 1205 of FIG. 13 (when the two are attached, they become an assembly, albeit a temporary assembly). In this exemplary assembly, the reservoir fill assembly 1205 is removably attached to the apparatus 1200. In this exemplary embodiment, consistent with the embodiments above, the reservoir fill assembly includes a septum configured to receive a termination of a syringe to enable a therapeutic substance to be delivered to the reservoir fill assembly and then into the reservoir of the apparatus 1200.

By “removably attached to the apparatus,” it is meant that the reservoir fill assembly can be readily removed from the apparatus 1200 without damaging the apparatus 1200 or otherwise physically altering a component of the apparatus 1200 in a manner that results in a configuration of the apparatus 1200 that is not an intended design configuration. For example, if the termination 1212 was instead a tube adhered to the tube 714 via an adhesive or a bonding material, so that the tube 1212 was permanently adhered to tube 714 and removing the tube 1212 would break the tube 1212 or the tube 714 or result in fracturing or breaking the adhesive (which was meant to be a permanent connection), that would be an example of something that is not removably attached to the apparatus.

Embodiments above have focused on the utilization of a septum or the like or some other device that enables a termination of a syringe to pass therethrough to reach the cistern, more accurately, the internal volume of the cistern. But embodiments can include another regime of accessing the internal volume of the cistern. In an exemplary embodiment, the reservoir fill assembly is configured so that a bayonet coupling or a bayonet adapter of a container of a therapeutic substance can be connected to the cistern, and thus the cistern has the corresponding coupling to receive the adapter of the container, and upon placing the container of therapeutic substance in the fluid communication with the interior of the cistern by coupling the latter to the former utilizing the adapters, the therapeutic substance can be transferred from the container to the cistern, and thus transferred into the reservoir of the implantable component. In an embodiment, a luer taper and/or luer lock can be used to connect to the cistern.

Accordingly, it can be seen that the reservoir fill assembly is a mechanism that enables convenience for the action of filling or charging the reservoir of the implantable component, such as because of the relatively large surface area of the septum by way of example. This is opposed to a fill assembly where, for example, there is only tube 1240 and the healthcare professional must insert a sharp or narrow termination into a relatively narrow opening in the tube or pierce the tube for example. And it is to be understood that in at least some embodiments, there will be a significant amount of waste owing to the cistern. In part, this can be because at least some embodiments related to the removable reservoir fill assembly utilizes the same cistern that is utilized in the implant detailed above. Embodiments can include a cistern that has a reduced internal volume, such as by way of example internal volume that is V-shaped with the pointed portion of the on the side opposite the septum. Indeed, in embodiments that utilizes the coupling, there might be no cistern that has a large internal volume. In this regard, the adapter could be located at the end of the tube 1240 so that the container holding the therapeutic substance can be bayonet coupled to the adapter, and the couplings being large enough so that these can be relatively easily manipulated by healthcare professional, but having internal volumes that are sufficiently small so that there is less waste of the therapeutic substance.

And note that in an exemplary embodiment, the therapeutic substance might “squirt” out of passageway 1212 when the reservoir is full. In an exemplary embodiment, the pressure buildup during the charging and filling could be such that once full, continued attempted transfer of the therapeutic substance in the reservoir will cause a pressure buildup that expands the silicone body 183 that extends about the termination 1250, that is, the portions of the body that are proximate the passageway 1212, thus permitting the therapeutic substance to flow backwards out around the termination 1250. This can give an indication to the healthcare professional that the reservoir is full. Further, this can provide a safeguard mechanism to avoid “bursting” or otherwise damaging the reservoir, such as by way of example, causing a sufficient increase in pressure that pushes the plug 530 out of the end of the tube 718. In this regard, other devices can be utilized to avoid this potential failure mode. By way of example, a pressure relief valve could be located on the reservoir fill assembly with the septum can be sufficiently flexible or otherwise of limited strength such that a pressure buildup within the cistern owing to the fact that the reservoir has become full results in the therapeutic substance squirting out around the termination of the syringe in between the termination and the septum.

And, in some embodiments, the plug 530 can be configured to be sufficiently porous that the increased pressure will result in therapeutic substance “leaking” out of the plug. Indeed, in an exemplary embodiment, this can be how the healthcare provider determines that the reservoir is full. In an exemplary embodiment of this embodiment, there might have to be some care taken to avoid increasing the pressure to the point where the leakage cannot keep up with the increase in pressure. In an embodiment, an orifice plate and/or some sort of internal restriction within the fill assembly can aid in limiting flow rate/pressure during charging, thus reducing (including eliminating) the leakage. In this regard, the syringe that is utilized to be a syringe that purposely provides resistance to the user so that the user can only transfer therapeutic substance at a rate that is lower than that which would cause damage to the reservoir. But again, embodiments can also include a sacrificial component for example on the reservoir fill assembly. Embodiments also include a digitally controlled (or an analogue controlled) fill/refill system. The system can control flow rate and/or control pressure so as to avoid the scenario of increasing pressure where there is leakage that is unacceptable or undesirable (in amounts at least).

Embodiments have focused on the concept of utilizing a syringe or some type of container that holds a therapeutic substance prior to transferring of the therapeutic substance to the reservoir fill assembly. In an alternate embodiment, the reservoir fill assembly can also be the container that holds a therapeutic substance. With regard to the former arrangement, in exemplary embodiments, the therapeutic substance is obtained from a traditional container, such as a glass container, that can have a septum at the top of the container. Concomitant with the traditional method of filling a syringe from a multi-dose bottle, the termination of the syringe is extended into the septum of the bottle and the desired amount of the therapeutic substance is withdrawn by pulling the plunger of the syringe backward. After the syringe is charged, the syringe is then utilized to charge the reservoir fill assembly. In an alternative scenario, single use pre-charged syringes with the therapeutic substance are obtained. The syringe, which already has the therapeutic substance therein, such as might be the case where the manufacturer delivers the therapeutic substance already in a given syringe, is then used to charge the removable reservoir fill assembly (e.g., by piercing the septum with the termination). Corollary to this is that many syringes do not have a termination per se. Instead, the end of the syringe, which can include a threaded body or a bayonet fitting for example, can be directly attached to another coupling of the device that is to receive the therapeutic substance. This is sometimes utilized for people who have a long-term port in their arm (e.g., for people who are taking daily doses of antibiotics for example—the container of antibiotics is essentially coupled to the adapter at the end of the tube that extends to the intravenous needle into the patient). This can be an example of the coupling arrangement detailed above. Instead of a cistern at the end of tube 1240, the typically female component (but could be male component) of the adapter is present at the end of tube 1240, which component is configured to be attached to the distal end of the syringe (e.g., by threading the distal end of the syringe into the female component, which also can be threaded, or snap fitted or bayonet coupled, etc.).

But all of the above embodiments just described relate to transferring the therapeutic substance from one container to the fill assembly. Embodiments include instead a fill assembly that is itself a therapeutic substance container with a delivery mechanism. FIG. 15 shows a reservoir fill assembly 1505 that utilizes at least some of the features detailed above, where there is a container 1510 that stores the therapeutic substance in a long-term manner in a way analogous to how the glass container with the septum could store the substance prior to utilizing a syringe to withdraw the therapeutic substance therefrom. The container 1510 is in fluid communication with tube 1240. Here, there is a cap 1560 at the end of termination 1250. This cap 1560 would be removed prior to insertion or attachment of the fill assembly 1505 to the implantable component 1200. In an exemplary embodiment, a portion of the container 1510 is frangible so that upon the application of a limited amount of pressure, a pressure barrier will break thus placing the interior of the container 1510 into fluid communication with tube 1240. Container 1510 could be a plastic flexible oval-shaped container that can be squeezed with one's hand or pinched with one's fingers to increase the pressure, and thus cause the therapeutic substance to move from the container and/or from the tube and/or from the termination into the removable component.

In an embodiment, the termination 1250 includes a frangible portion (when the portion “breaks” the portions are still held to the termination (there can be weakened areas that split from each other)) where pressure breaks open the portion so that the substance can be moved out of the fill assembly 1505. In an embodiment, a plug in the termination 1250 could be punctured with a needle, or the plug could be cut off to “open”the termination.

Thus, in an embodiment, there is an assembly that includes the apparatus 1200 for example, and a reservoir fill assembly such as reservoir fill assembly 1505, where the reservoir fill assembly is removably attached to the apparatus, as would be the case prior to implantation of the apparatus 1200 by way of example. This reservoir fill assembly 1505 is removably attached to the apparatus 1200. The reservoir fill assembly includes a therapeutic substance, and the reservoir fill assembly is configured so that an internal volume thereof containing the therapeutic substance can be reduced, thereby driving the therapeutic substance into the reservoir. And while the embodiment shown in FIG. 15 describes a collapsible container, in another embodiment, element 1510 can be a syringe where the entire assembly is delivered to the healthcare professional from the therapeutic substance manufacturer in this form.

In some embodiments, the reservoir could be charged utilizing a termination of a hypodermic syringe by way of example, or more specifically, instead of the aforementioned self-healing septum on the cistern, a portion of the tube 714 can be such that the tube could be pierced by the termination. With reference to FIG. 11, element 930B could instead be a target that is printed or otherwise present on the outer surface of the body 516. The healthcare provider could insert the termination of the syringe through the body 516 to reach the interior of the tube 714. Upon sufficient insertion, the therapeutic substance can be delivered to the tube 714. Alternatively, and/or in addition to this, element 930B could be a funnel like device that funnels the tip of the termination to the tube 714 to decrease the likelihood that the tube would be missed by the healthcare provider. Of course, such embodiments run the risk of having the healthcare provider pierce both sides of the tube. An embodiment could have a portion of the tube that is titanium or the like or some other relatively strong body that will prevent the termination from passing through both sides of the tube. An embodiment could be such that the termination and 930B could be configured to interact but not pierce, such as, for example, there could be a blunt termination that abuts element 930B, or more accurately, an opening therein. This could be akin our could be a bionector needle free valve by way of example.

As with the embodiment associated with the passageway 1212 above, the body 516 be such that the passageway created by the termination would then collapse upon the termination being removed, thus fluidly ceiling or otherwise preventing the movement of the therapeutic substance out of tube 714.

In an embodiment, there is an apparatus such as implant 1200 that is configured to removably receive a termination (e.g., a tube or syringe needle) of a therapeutic substance charging device (device 1505 or device 1205 by way of example) to enable therapeutic substance located in the charging device to be delivered to the reservoir of the implant 1200. In this embodiment, the apparatus is configured to seal the reservoir upon removal of the tube prior to implantation of the apparatus into a human so that the therapeutic substance will not escape the reservoir. In an embodiment, the collapsed passageway may permit a de minimus amount of the substance to escape, such as in the event of a pressure gradient. Note that this could be the case with other seal embodiments as well (permitting a de minimums amount to escape).

In an embodiment, passageway 1212 can be a fill port. As noted above, in an embodiment, the fill port is a collapsible gap in silicone of the implantable portion, which gap collapses upon removal of a fill termination placed in the collapsible gap to seal the reservoir (or at least limit leakage) so that the therapeutic substance will not escape the reservoir.

Thus, in an embodiment, there is an assembly that includes an implantable portion and includes a reservoir fill assembly in fluid communication with the reservoir via the fill port, wherein the reservoir fill assembly is configured to be at least substantially filled with a therapeutic substance to fill the reservoir, and the reservoir fill assembly is removable from the fill port so that the apparatus can be implanted without the reservoir fill assembly. In an embodiment, upon the removal of the reservoir fill assembly from the fill port, the implantable portion is ready to be implanted in the recipient at least with respect to actions related to closing the fill port, because in this embodiment, the fill port is self-sealing and/or self-closing. That said, in an alternate embodiment, the fill port could be “filled” with another substance, such as a quick curing silicone or an adhesive, or otherwise such substance could be injected or otherwise placed into the passageway, to further enhance the sealing fixtures. In an embodiment, the another substance used to fill the port does not permit the substance to diffuse, unlike the diffusion port material for the outlet. To the extend that there is diffusion, it is far far lower than the diffusion of the diffusion port material for the outlet.

In an embodiment, compression can be applied after the termination is removed or otherwise a component can be located in the silicone body, such as a spring or the like, that will permit the termination to open the passageway when the termination is pressed towards the tube, but then once the termination is removed, the component will compress the silicone to close the passageway. That said, the spring or the like need not necessarily be present. Instead, a C-shaped body that is relatively rigid or solid could be located on either side of the passageway, where the simple fact that the silicone fills the interior of the seal is sufficient to result in the self-healing or otherwise closure the passageway when the termination is removed. That is, the termination will compress the silicone against the sides of the C-shaped body (the interior of the C-shaped body), thus opening up the passageway, and then when removed, the silicone will expand back to its original status, and thus close the passageway.

In an embodiment, removal of the reservoir fill assembly reduces the volume of therapeutic substance accessible to the reservoir by at least 30, 35, 40, 45, 50, 55, 60, 65, 70, 75, 80, 85, 90, or 95% or more, or any value or range of values therebetween in 0.1% increments. In this regard, as would be understood by some of these numbers, the reservoir fill assembly has in some embodiments a larger volume than the reservoir of the implantable component. In this regard, when the assembly of the implantable component in the reservoir fill assembly are together with the reservoir, there is a combined first volume which first volume is the combination of the reservoir plus a second volume which second volume is the reservoir fill assembly, and potentially a third volume which is a portion of the passageway 1212 if the termination 1250 does not reach the tube 714. In this regard, it is noted that the passageway 1212 is not part of the reservoir. There could be residual therapeutic substance located in the reservoir upon the removal of the termination 1250. However, that is not part of the reservoir. In this regard, the reservoir stops at the opening of the tube 714 or otherwise the adapter on the tube 714. In this embodiment, upon removal of the termination, and the subsequent collapse or otherwise sealing of the passageway 1212, the volume of therapeutic substance accessible to the reservoir is reduced by one or more of the values just detailed.

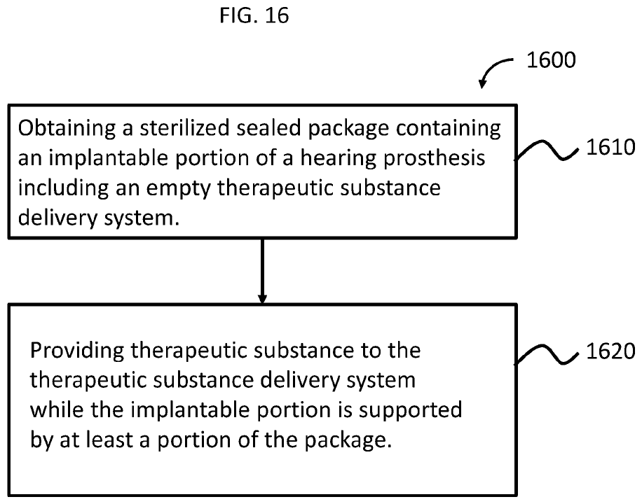

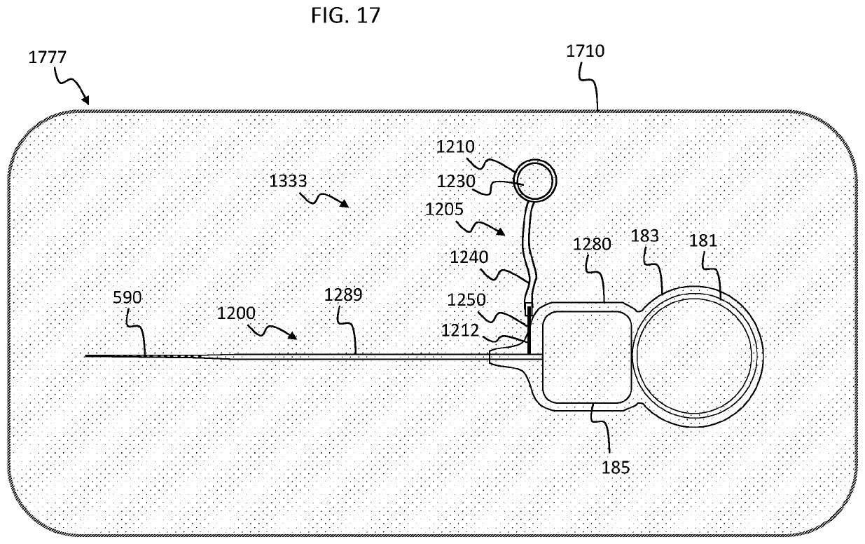

Embodiments include methods. FIG. 16 presents an exemplary flowchart for an exemplary method, method 1600, according to an exemplary embodiment, method 1600 includes method action 1610, which includes the action of obtaining a sterilized sealed package containing an implantable portion of a hearing prosthesis including an empty therapeutic substance delivery system (e.g., the reservoir with the plug 530 as detailed above). FIG. 17 shows an exemplary embodiment of an implantable portion of a hearing prosthesis 1200 including an empty therapeutic substance delivery system. Also shown in this embodiment is the fill assembly 1205. Collectively, this establishes an assembly 1333. Collectively, these components are located in a sterilely sealed package 1710, which collectively establishes an assembly 1777. The package can be made of plastic or the like, that is in some embodiments vacuum sealed or in other embodiments he shrunk about the components of the implantable portion and the fill assembly. In an embodiment, the base of the package includes formed recesses that except at least a certain amount with respect to height (the direction in and out of the page 17) of the components shown. A lid or a cover (represented by the dotted shaded portions of the package 1710) which in some embodiments, at least a portion thereof that is transparent or semitransparent, is placed over the components to be sealed in the package and then the cover is sealed to the base to “trap or otherwise maintain the components in the package 1777.

It is briefly noted that this is just one exemplary embodiment of packaging and one exemplary embodiment of the components that are in the package. Other embodiments can include other features as detailed above or other variations providing that the art enables such.

Referring back to the flowchart of FIG. 16, embodiments include method action 1620, which includes the action of providing therapeutic substance to the therapeutic substance delivery system while the implantable portion 1200 is supported by at least a portion of the package. (Briefly, it is noted that the fill assembly 1205 is not part of the therapeutic substance delivery system per se. The fill assembly is simply a way to enable the therapeutic substance delivery system to be charged or otherwise provided with therapeutic substance. The therapeutic substance delivery system refers to the arrangement of the implantable portion that enables a therapeutic substance to be delivered to a recipient to have utilitarian value.) In an exemplary embodiment, prior to method action 1620, the cover or lid is peeled off or otherwise removed from the base of the package 1777. This eliminates the sterile seal if present. But this provides or otherwise provides access to the assembly 1333. In an exemplary embodiment, while the assembly 1333, or at least the implantable portion 1200, is supported by the base (e.g., in the affirmation recesses that are formed in the plastic or the polymer base in this exemplary embodiment) a termination of a syringe is inserted through the septum 1230, and then therapeutic substance is injected into the cistern 1210, and then the therapeutic substance flows into the reservoir of the implantable portion 1200, thus executing method action 1620. In an embodiment, there is an indicator, much like the EO (or EtO) exposure indicators, where there is an apparatus on/in the packaging that absorbs and/or spreads the first droplet out of the cannula to make it very visually certain that the device is loaded with therapeutic substance.

In an alternate embodiment, the seal or cover is sufficiently puncturable or otherwise of a design that enables the healthcare professional or the like to place the termination of a syringe through the cover to reach the septum 1230 and puncture the septum. In this way, the method action 1620 can be executed without opening the package 1777. Thus, in an embodiment, the action of providing therapeutic substance to the therapeutic substance delivery system is executed while the implantable portion is still sterilely sealed within the package.

In an exemplary embodiment, the components in the package, or at least the implantable portion 1200, remain sterilely sealed in the package. In an exemplary embodiment, the cover of the package can be configured so that the package self-heals. In an exemplary embodiment, a septum can be built into the package, which septum is self-healing. In this regard, the action of transferring therapeutic substance from the syringe to the cistern 1230 by puncturing the septum that is built into the package can be analogous to utilizing a termination of a syringe to puncture a septum of a container of therapeutic substance to charge the syringe. In an embodiment, instead of remaining sterilely sealed, the implantable portion 1200 remains at least largely covered. This can have utilitarian value in that most bacteria “fall” onto implants. Thus, even if there is a small opening to allow tube 1240 through, the portion 1200 can be “covered” from the vast majority of airborne pathogens. Thus, in an embodiment, during charging/filling of the therapeutic substance while the portion 1200 is in the packaging or otherwise supported by the packaging, at least 85, 86, 87, 88, 89, 90, 91, 92, 93, 94, 95, 96, 97, 97.5, 98, 98.5, 99, 99.1, 99.2, 99.3, 99.4, 99.5, 99.6, 99.7 percent or more or any value or range of values therebetween in 0.01% increments of the surface area facing away from the direction of gravity during charging (which can be represented by a silhouette looking downward) is covered by material of the packaging (there is packaging between the portion 1200 and the above/a location 1 or 2 or 3 inches directly above the silhouette).

The point is that in at least some exemplary embodiments, the arrangement of FIG. 17 enables the implantable portion 1200 to be prepared for implantation prior to removing the implantable portion 1200 from the package, or at least from the base. In an exemplary embodiment, the healthcare provider then removes the implantable portion 1200 which is now charged with therapeutic substance from the base and then removes the fill assembly 1205 from the implantable portion 1200, and essentially immediately proceeds to implant the implantable portion 1200 in a recipient. In this regard, there are no or minimal intermediate actions between the action of removing the implantable portion 1200 from the packaging and implanting the implantable portion in the human. This as opposed to, for example, charging the reservoir of the implantable portion 1200 after the implantable portion 1200 is removed from the package.

In an embodiment, the fill assembly 1205 is configured to be retained in the package. That is, upon the surgeon or healthcare professional removing the implantable portion 1200 from the base of the package, the termination 1250 will be pulled out of the silicone body 183 because the fill assembly 1205 will be retained to the base. That said, in an exemplary embodiment, the fill assembly is also removable, and the surgeon removes the fill assembly from the implantable portion 1200 upon removing the assembly 1333 from the packet 1777 or otherwise the base thereof.

Thus, in an exemplary embodiment, the action of providing therapeutic substance is executed by injecting the therapeutic substance into a temporary fill device (e.g., fill device 1205) connected to the implantable portion and fluidically connected to the therapeutic substance delivery system at the time that the package is obtained. In this embodiment, the method further comprises removing the temporary fill device after providing the therapeutic substance and implanting the implantable portion in a human without the temporary fill device.

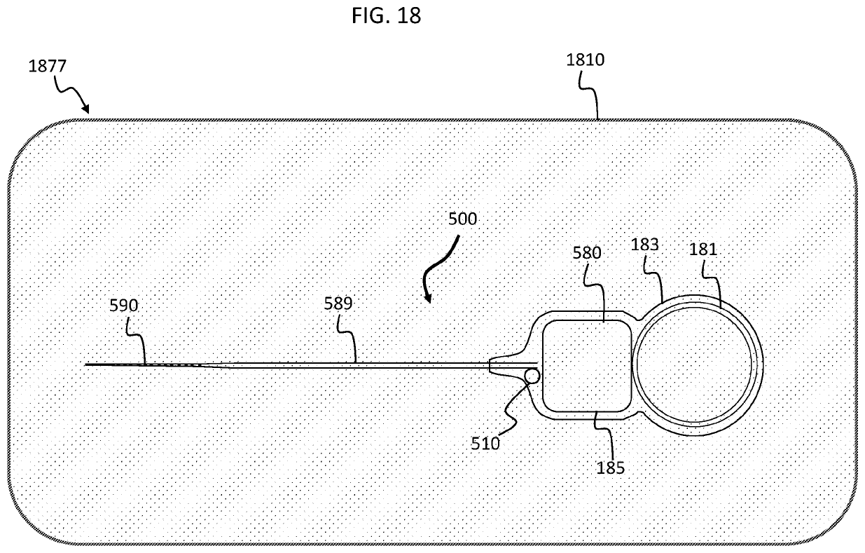

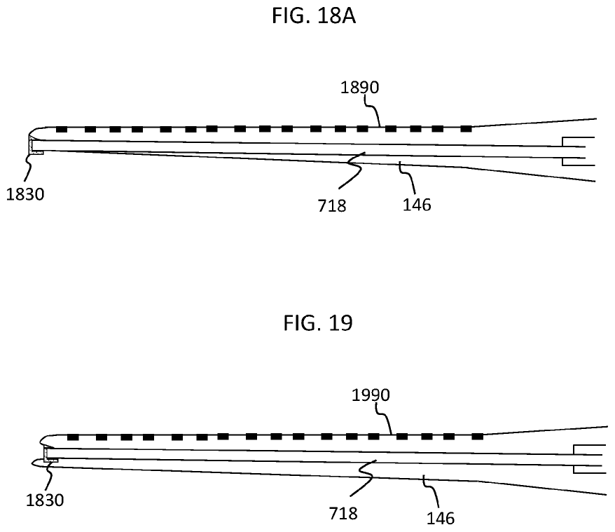

And note that in other embodiments different from the method 1600 just detailed, the entire assembly 1333 can be removed from the package 1777, and not supported by the base, and then the reservoir can be charged with therapeutic substance accordingly. Note further that embodiments include packaging that only has the implantable portion 1200 that is sterilely sealed in the package. FIG. 18 presents an exemplary embodiment of such, where assembly 1877 includes the implantable portion 500 sealed in package 1810, where the base has a formed recess for the implantable assembly 500. In a manner concomitant with the teachings above, a termination of a syringe can pierce the cover of the package 1810 to reach the cistern 1510, which in this exemplary embodiment, has a self-healing septum facing the viewer of FIG. 18, and thus the termination pierces the self-healing septum to fill the reservoir of the implantable portion 1500 with therapeutic substance. Alternatively, this can be done after the cover is removed from package 1810, and thus while the implantable portion 500 is still supported by a portion of the package.

In an embodiment, the septum of the package can include an antibiotic and/or antiviral coating or substance that sterilizes the termination as it passes through the septum and/or that prevents bacteria from settling and/or forming colonies thereon. In an embodiment, there is a sterilizing fluid (liquid and/or gas) located between the cover the package and/or the septum of the package, and the septum of the fill assembly. In an exemplary embodiment of this embodiment, when the termination of the syringe punctures the septum where the cover, the termination then punctures a container locating the sterilizing fluid, which then sterilizers any contamination that might result from the action of puncturing the septum and/or the cover. After the cover is ultimately opened, any residual fluid could be cleaned off of the implant. Alternatively, and/or in addition to this, the package could have a container that could be broken from the outside irrespective of the use of the termination, so that the fluid can flow about the areas to be re-sterilized if there is some contamination.

Embodiments include obtaining a package with the implantable portion of the implant sterilely sealed therein, where the therapeutic substance is located in the sterilely sealed package when the package is obtained. In this exemplary embodiment, the therapeutic substance is shipped or otherwise provided with the cochlear implant in one package. The therapeutic substance is simply separated from the implant, or otherwise the reservoir is not charged with the therapeutic substance. In an exemplary embodiment, the therapeutic substance delivery assembly can be the bolus detailed above where if the bolus is squeezed, the therapeutic substance can be delivered to the reservoir, and thus the reservoir can be charged. In this embodiment, this could be done by way of example without opening or even puncturing the package.

In an embodiment, the fill assembly can include a syringe that is sealed within the package, where the syringe can be actuated through the material of the package.

Thus, embodiments include executing method action 1600 where the therapeutic substance is located in the sterilized sealed package when the package is obtained. In an exemplary embodiment, the action of obtaining the sterilely sealed package occurs when the package is provided to the healthcare professional or an agent of the healthcare professional (e.g., a nurse or assistant who is preparing the medical device for implantation just prior to surgery) who is going to execute the implantation procedure of the implantable portion within the package. In an exemplary embodiment, exemplary methods include obtaining a package that includes the implantable portion and optionally the therapeutic substance sterilely sealed within the package from storage within a hospital or some other surgical center that is qualified to implant the implantable portion in a human in a surgical procedure. In an exemplary embodiment, the package and thus at least the implantable portion has been stored at the center for at least 1, 2, 3, 4, 5, 6, 7, 8, 9, 10, 11, 12, 13, 14, 15, 20, 25, 30, 35, 40, 45, 50, 55, or 60 weeks and/or 15, 20, 25, 30, 35, 40, 45, 50, 55 or 60 months or any value or range of values therebetween in one week increments.

In an exemplary embodiment, the action of charging the reservoir as detailed herein occurs within 24, 18, 12, 6, 5, 4, 3, 2, or 1 hour, or any value or range of value therebetween in one minute increments prior to placing at least a portion of the implantable portion inside the body of a recipient through an artificially created opening in the skin of the recipient. In an embodiment, the action of charging the reservoir as detailed herein occurs within 90, 80, 70, 60, 50, 40, 30, 25, 20, 15, 14, 13, 12, 11, 10, 9, 8, 7, 6, 5, 4, 3, 2, or 1 minutes or any value or range of value therebetween in 1 second increments prior to placing at least a portion of the implantable portion inside the body of a recipient through an artificially created opening in the skin of the recipient. In an exemplary embodiment, the portion that can be placed into the recipient could be the receiver-stimulator when it is placed over the mastoid bone through the incision of the skin of the head over the mastoid bone.

Thus, embodiments provide a regime of separating the therapeutic substance from the implantable portion up to the point of surgery. Note also that embodiments include charging the reservoir after the at least a portion of the implantable portion is placed in the human. In an exemplary embodiment, this could be the very last thing that is done. For example, the implantable portion can be placed inside the recipient with the fill assembly attached, and then the fill assembly can be used to charge the reservoir, and then the fill assembly could be removed and then the surgery completed by closing the opening.

Embodiments thus include the action of implanting the implantable portion in a human after providing the therapeutic substance. In an embodiment, no more therapeutic substance is provided to the therapeutic substance delivery system after the action of providing therapeutic substance. In this exemplary embodiment, this can correspond to a one time charging, where, after implantation, no more additional therapeutic substance is provided to the reservoir otherwise the implantable portion. That said, as noted above, embodiments can include, after implantation, utilizing a termination of a syringe to pierce the skin over the septum of the implantable cistern to recharge the therapeutic substance or to provide a different therapeutic substance after a certain amount of time.

Corollary to this is that embodiments include, prior to charging or otherwise filling the reservoir, selecting a particular type of therapeutic substance to be delivered by the implantable portion or otherwise to be placed into the reservoir. Embodiments thus can enable a wide variety of therapeutic substances to be selected at the time of surgery or within any of the after mentioned times associated with the first portion of the implant being located in the human. For example, therapeutic substance A could be selected for some patients and therapeutic substance B could be selected for other patients, all which could be selected at or during the surgical procedure. In an embodiment, the therapeutic substance is dexamethasone. But it is noted that other types of therapeutic substance can be used/contained in the reservoir, for example, systemic steroids, anticoagulants, clot busters, antifibrotics, antiproliferatives or NSAIDs. Therapeutic substances include drugs, but also include nondrug substances. In an exemplary embodiment, therapeutic substances include steroids (as just noted and/or biologics). Therapeutic substances can also include minerals and the like. Any disclosure herein of drug or the containment of drug or the delivery of drug also corresponds to another embodiment that corresponds to an embodiment that is directed towards a therapeutic substance. That is, typically, the word drug used herein is shorthand for therapeutic substance. Accordingly, embodiments include the present disclosure where the word drug is replaced by the word therapeutic substance, unless otherwise specified.

The therapeutic substance may be a corticosteroid such as betamethasone, clobetasol, diflorasone, fluocinolone, triamcinolone, salt, ester, or combination thereof.

In an embodiment, there is an apparatus, such as the implantable portion of the cochlear implant, or the lead assembly thereof, which includes an implantable electrode array including a plurality of electrodes supported by a silicone body. In this exemplary embodiment, the electrode array includes a therapeutic substance delivery channel (e.g., tube portion 518 or tube 718) including at least one port (e.g., the distal end portion of portion 581 or tube 718). In an exemplary embodiment, the port includes a distinct barrier made of a material that maintains a bacterial barrier prior to charging the channel with a water-based substance. In an exemplary embodiment, the barrier is plug 530. FIG. 18A presents an alternate exemplary embodiment of the distinct barrier with respect to electrode array 1890. Here, element 1830 is a cap that fits over the distal end of the tube 718 (instead of in the tube as with the plug). In this exemplary embodiment, tube 718 extends outward a tiny amount so that the cap can fit over the tube. In the embodiment shown in FIG. 18A, the cap 1830 is a forward surface of the array. In some embodiments, the cap protrudes somewhat from the silicone body that is the silicone carrier 146 that envelops tube 718. Conversely, FIG. 19 shows an exemplary electrode array 1990 where the material of the carrier 146 extends past the cap 1830. This can have utilitarian value with respect to protecting the cap otherwise providing that the most forward surface is the relatively flexible and soft silicone of the body that establishes the carrier 146. Any arrangement that can enable the teachings detailed herein can be utilized in at least some exemplary embodiments.

In an embodiment, the distinct barrier has pores. For example, the pores of the distinct barrier are no greater than 1, 0.9, 0.8, 0.7, 0.6, 0.5, 0.4, 0.35, 0.3, 0.25, 0.2, 0.15, 0.1, 0.09, 0.08, 0.07, 0.06, 0.05 microns, or any value or range of values therebetween in 0.005 micron increments (but note that some of these might not establish a bacterial barrier-some embodiments do not establish a bacterial barrier).

The barrier can be PVDF. Note that micropores or millipores can be used depending on the embodiment.

By distinct barrier, it is meant that the barrier is a different component in material and/or dimension and/or arrangement from the other components immediately adjacent the barrier. For example, the barrier is a different component in material and dimensioned from the silicone that establishes the carrier. The barrier is also different in material and dimension from the tube 718. In an embodiment, a rolled silicone filter could be used, or a porous silicone could be used.

In an exemplary embodiment, the distinct barrier is entirely made of polyvinylidene fluoride and can be made of Durapore®.

In an embodiment, the distinct barrier is configured to diffuse dexamethasone at a rate of no more than 30, 29, 28, 27, 26, 25, 24, 23, 22, 21, 20, 19, 18, 17, 16, 15, 14, 13, 12, 11, 10, 9.5, 9, 8.5, 8, 7.5, 7, 6.5, 6, 5.5, 5, 4.5, 4, 3.5, 3, 2.5, 2, 1.75, 1.5, 1.25, 1, 0.9, 0.8, 0.7, 0.6, 0.5, 0.4, 0.3, or 0.2 nanograms/hour (mean, median, and/or mode and/or maximum rate over any one or more of the time periods detailed herein) or any value or range of values therebetween in 0.05 nanogram/hour increments after implantation into a body, such as a body cavity, such as a human cochlea, at a pressure of 1 atmosphere plus or minus 0.1 atmosphere and/or at a pressure in the reservoir that results from implantation in the human after the surgery is completed, where the reservoir was charged at 1 atmosphere. (This does not mean that the charging must be done at 1 atmosphere. This means that when done at 1 atmosphere, this is what happens.) In an embodiment, the therapeutic substance diffuses through the barrier and is replaced with NaCL. As the concentration of the therapeutic substance decreases over time, so will the release rate.

In an embodiment, the distinct barrier is also configured so that an over-pressure in the channel relative to an outside environment will cause dexamethasone to be driven out the plug while maintaining the plug for subsequent use as a diffusion distinct barrier. In an exemplary embodiment, the over-pressure can be 10, 15, 20, 25, 30, 35, 40, 45, 50, 55, 60, 70, 80, 90, 100, 125, 150, 175, 200, 250, 300, 350 or 400% or more or any value or range of values there between in 1% increments where the denominator is the pressure prior to the overpressure. This can have utilitarian value with respect to a scenario where an overpressure exists during the charging operation the reservoir. The idea being that it is utilitarian for the cap or plug to not “pop” off or rupture. In an embodiment, the tube(s) of the reservoir also are configured so that an overpressure in the reservoir maintains the reservoir for subsequent use when implanted in a human. In an embodiment, there is a valve that is configured to eject substance upon overpressure. This can be located along the lead and/or at the cistern. This can be located away from the electrode array/portions that are located/to be located in the cochlea upon full implantation. In an embodiment, there can be a poppet that pops out upon over pressurization. The poppet can be configured so that it must be replaced or a new one is placed at the location of the old one. Spare poppets can be provided with the implant in the packaging. In an embodiment, the valve/poppet ensures or otherwise reduces the likelihood of damage to the substance delivery system via over pressurization. In a sense, the poppet can be a sacrificial component.

In an embodiment, there is little to no mass transfer when the therapeutic substance is delivered to the recipient. All delivery or substantially all delivery is executed by diffusion.

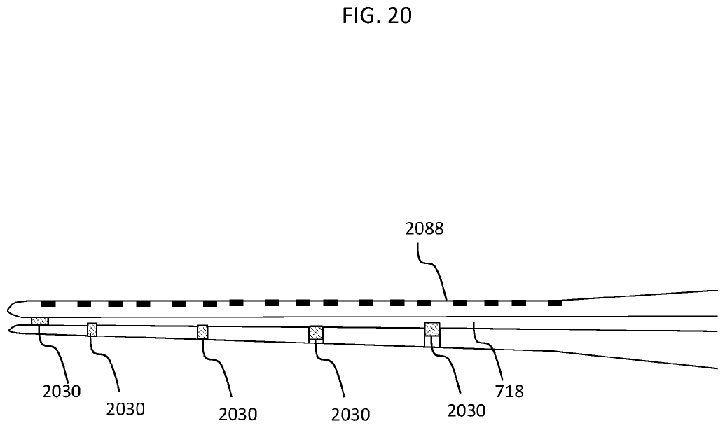

While the embodiments above have focused on a single outlet for the therapeutic substance, embodiments include reservoirs that have multiple outlets. In this regard, FIG. 20 shows an exemplary electrode array 2888 that includes outlets located on the lateral wall facing side of the electrode array. Each of the outlets includes plugs 2030, which can correspond to the plugs detailed above. Also shown is the various placement of the plugs. As can be seen, some of the plugs can be located in board of the most outboard portions of the outlet, and some of plugs can extend into the tube 718. Providing that the therapeutic substance can transfer from the tube to the ambient environment after implantation, any arrangement can be utilized. It is also noted that the outlets can have various sizes and/or dimensions and they can be different from one another. In an exemplary embodiment, the plugs 2030 are adhesively bonded to the tube 718 and/or to the silicone body that establishes the carrier. In this embodiment, the tube 718 has orifices located along the length thereof, and the plugs 2030 fill the orifices. In an embodiment, the plugs interference fit with the orifices two maintain the plugs in the orifices. In an embodiment, again adhesive or some other bonding technique is utilized.

In an exemplary embodiment, the reservoir is a non-expandable reservoir. By way of example only and not by way of limitation, in an exemplary embodiment, the reservoir is might be made out of an elastomeric material, but the reservoir is structured so that the elasticity is de minimus. In an exemplary embodiment, in a 1 atm pressurize state, the reservoir establishes a first interior volume. When subjected to a pressurization such as any one or more of the pressurization's above by way of example, depending on the pressurization, the reservoir establishes a second interior volume that is no more than 10, 9, 8, 7, 6, 5, 4, 3, 2 or 1% larger than the first interior volume.

It is noted that the phrase “filled” as used herein is not an absolute term. This refers to the action of placing the substance into the reservoir from a location outside of the reservoir. The reservoir need not be filled to capacity. Although embodiments do include filling the reservoir to capacity.

Some embodiments of the therapeutic substance can be a drug. Thus, embodiments are directed towards a drug delivery system. Therapeutic substances include drugs, but also include nondrug substances. In an exemplary embodiment, therapeutic substances include steroids and biologics. Therapeutic substances can also include minerals and the like.

In an exemplary embodiment, the barriers described above serve a dual function as a flow restrictor and bacterial barrier.

In view of the above, as can be seen, in an exemplary embodiment, there is an apparatus, comprising, by way of example, a cochlear implant electrode array, and an implantable drug reservoir. In this exemplary embodiment, the apparatus is configured such that the drug reservoir is part of the electrode array, and the drug reservoir is at least substantially located outside of the middle ear space and outside of the inner ear space when the cochlear implant electrode array is fully implanted in a recipient. (Note that reservoir is a relative term. Because of the relative de minimis size of the cistern, the tubing of the lead assembly including the electrode array is considered a reservoir. Conversely, if the cistern was much larger than at least some of the exemplary embodiments disclosed herein, the tubes of the lead assembly would not be considered reservoirs.) In the embodiments above, the cistern is totally located outside the middle ear and the inner ear when the cochlear implant electrode array is fully implanted in a recipient, both in the relaxed state and in the fully operational expanded state. In at least some embodiments, the cistern is an inelastic enclosure aside from the septum or a totally inelastic enclosure.

In an exemplary embodiment, in a 1 atm pressurize state, the cistern establishes a first interior volume. When subjected to a pressurization such as any one or more of the pressurization's above by way of example, depending on the pressurization, the reservoir establishes a second interior volume that is no more than 20, 19, 18, 17, 16, 15, 14, 13, 12, 11, 10, 9, 8, 7, 6, 5, 4, 3, 2, or 1% larger than the first interior volume. In an embodiment, if the septum is rigidly retrained to avoid expansion, the reservoir establishes a second interior volume that is no more than 10, 9, 8, 7, 6, 5, 4, 3, 2, or 1% larger than the first interior volume.

It is noted that the barriers described above do not elute the drug or therapeutic substance, or more accurately, that is not the principle of drug delivery (more on this in a moment), but instead can diffuse the substance. In an embodiment, the substance does not pour out, but instead worms its way through the porous barriers. In an embodiment, only considering the diffusion-based delivery, the barrier is not needed at all—the outlet could be open. The barrier has pores allowing direct fluidic connection between the liquid inside (drug solution) and the liquid outside (perilymph) the therapeutic substance delivery system. This allows free diffusion of the therapeutic substance molecules from inside the device to outside across the barrier following a concentration gradient. That is, the therapeutic substance molecules do not need to dissolve or absorb or adsorb into a third matrix (considering the therapeutic substance solvent inside the device as a first matrix and perilymph (or other bodily fluids) as the second matrix). The barrier is utilized to prevent pathogens such as virus, bacterium, protozoan, prion, viroid, and/or fungus to exit and enter the device (e.g., a bacterial filter of 0.22 micrometer pore size or smaller is used in some embodiments). The barrier also provides, in some embodiments, a mechanical mechanism to increase the flow resistance between the lumen inside the device and the outside environment (i.e., perilymph in the implanted state). This can enable containment of the therapeutic substance solution inside the device after initial priming (filling) during handling and implanting the implant. In the implanted state the barrier's flow resistance helps to avoid significant amounts of liquid to exit or enter the delivery system in the event of pressure changes inside or outside the device from, for example, body movement or impact, or simply handling of the device during implantation.

In an embodiment, there could be some de minimums diffusion and/or elution of the therapeutic substance through the tubes and/or through the silicone body of the lead assembly. In at least some exemplary embodiments, this is unwanted, but because of the limited amounts at play, does not present a problem. In an exemplary embodiment, of the therapeutic substance that is used to charge or fill the reservoir, no more than 10, 9, 8, 7, 6, 5, 4, 3, 2, 1, 0.5, 0.25, or 0.1% or any value or range of values therebetween in 0.1% increments of the therapeutic substance diffuses and/or elutes through the tubes and/or the silicone bodies. It is noted that some of the aforementioned values may not be desired or tolerable, while in other embodiments they may be tolerable. In an exemplary embodiment, the rate of delivery is at least substantially based on diffusion through the distinctive barrier. In an exemplary embodiment, of the therapeutic substance that leaves the reservoir and enters the body, at least 90, 91, 92, 93, 94, 95, 96, 97, 98, 99, or 100%, or any value or range of values therebetween in 0.1% increments of such is a result of diffusion, including diffusion through the distinct barrier, within any one or more of the timeframes detailed herein (or in total).

In an embodiment, the therapeutic substance is water-based, and the therapeutic substance diffuses out while the water remains in the delivery system and salts, etc., diffuses into the reservoir to address the concentration gradient. In an embodiment, net water movement into the device (osmosis) is avoided. In embodiments, a therapeutic solution that is isotonic to match the osmolarity of perilymph to avoid osmosis is utilized.

This is contrasted to, for example, the operation of the membrane, where the therapeutic substance or otherwise the active ingredient, actually comes out of the water, and then passes into the membrane, and then passes back into the water of the cochlea for example. Embodiments according to the teachings detailed herein are such that the therapeutic substance does not leave the water that is located in the reservoir at the time of charging with the therapeutic substance. The diffusion is a diffusion of the therapeutic substance, as opposed to the principle of operation of the membrane.

In an exemplary embodiment, with respect to a given volume of the distinct barrier that establishes the filter between the reservoir and the ambient environment, the volume is at least 0.5, 0.75, 1, 1.25, 1.5, 1.75, 2, 2.25, 2.5, 2.75, 3, 3.5, 4, 4.5 or 5 percent porous (the remainder the material of the barrier) or any value or range of values therebetween in 0.01% increments.

Embodiments include utilizing different porosities and/or different dimensions of the discrete barrier to obtain different release rates. In an exemplary embodiment, a thicker or longer plug will result in a slower release rate than a thinner or shorter plug (these dimensions being related to the longitudinal axis of the electrode array).

In some embodiments, there still is exchange of the same amount of water molecules in and out across the filter, but to make up for the drug diffusing out (outflux) into perilymph, it can be assumed that it is not water molecules moving in but solutes from perilymph such as sodium and chloride diffusing in (influx) to keep the osmolarity the same. Accordingly, in an embodiment, the therapeutic substance solution is water-based, and the therapeutic substance stays dissolved in water, and diffuses out of the reservoir through open pores in the barrier into perilymph (outflux) following a gradient of a higher therapeutic substance concentration in the water-based therapeutic substance solution to a lower therapeutic substance concentration in the water-based perilymph.

In an embodiment, a total water amount of the therapeutic substance delivery system at the time of implantation is within 15, 14, 13, 12, 11, 10, 9, 8, 7, 6, 5, 4.5, 4, 3.5, 3, 2.5, 2, 1.5, 1, 0.9, 0.8, 0.7, 0.6 or 0.5% or any value or range of values therebetween in 0.05% increments of the value of water amount at the point when 80, 85, 90 or 95% of the therapeutic substance has diffused out of the system into the body.

In an embodiment, there is no pressure gradient that causes the therapeutic substance to diffuse or otherwise leave the reservoir, at least after the implantable component is implanted in a human. Indeed, in an exemplary embodiment, the distinctive barriers herein, such as the filters, damping pressure changes. In an exemplary embodiment, the systems are configured to avoid in overpressure (or under pressure by any one or more of the amounts detailed herein. As briefly noted above, in some embodiments, the pressure of the initial charging will cause the therapeutic substance to be out or otherwise leave the barriers, and this can be utilized to determine whether or not the reservoir is full or otherwise that the therapeutic substances reached the tip of the electrode array, but after the charging process, the pressure inside the reservoir should be approximately room pressure or whatever the is the local ambient pressure. That is, the reservoir is not a pressurized system. The principle of operation of therapeutic substance delivery occurs from the concentration gradient that exists inside the reservoir with respect to the therapeutic substance relative to the therapeutic substance outside the reservoir, specifically, the cochlea in the embodiment where the devices a cochlear implant electrode array. In this regard, initially, such as within minutes of insertion of the electrode array into the cochlea, the barrier will be in fluidic contact with the perilymph of the cochlea or other bodily fluid with respect to another body cavity. Thus, there will be a gradient between the outside the reservoir inside of the reservoir relative to the distinct barrier. Over time, this gradient will fall to a 1 to 1 ratio or substantially a 1 to 1 ratio, owing to the “desire” of the system to balance the chemical state inside the reservoir to the outside of the reservoir. In an exemplary embodiment, within 1, 2, 3, 4, 5, 6, 7, 8, 9, 10, 11, 12, 13, 14, 15, 16, 20, 25, 30, 35, 40, 45, 50, 60, 70, 80, 90, 100, 125, 150, 175, 200, 250, 300, 350, 400, 450, 500, 550, 600, 700, 800, 900, 1000, 1250, 1500, 1750, 2000, 2250, 2500, 3000, 3500, 4000, 4500 or 5000 hours after first entering the cochlea/the barrier coming into contact with the perilymph or pertinent body fluid, at least and/or no more than 50, 55, 60, 65, 70, 75, 80, 85, 90, 91, 92, 93, 94, or 95%, or any value or range of values in 1% increments of the therapeutic substance that was in the reservoir at the time that the distinct barrier came into contact with the body fluid remains in the reservoir owing to diffusion through the distinct barrier into the cochlea or pertinent body cavity or body space.

In an exemplary embodiment, the therapeutic substance delivery system is a valveless system and/or a flow restrictor less system (save for the substantial barrier(s) to the extent they are considered flow restrictors.

In an exemplary embodiment, no portion of the lead assembly is saturated and/or no portion of the implantable portion is saturated with a therapeutic substance, with the possible exception of the substantial barrier or at least a portion of the substantial barrier in at least some exemplary embodiments.

In an embodiment, the silicone of the lead assembly and/or any of the tubes, or otherwise the material of the tubes, is not porous and/or is not aerated.

As seen above, in some embodiments, the reservoir and the lead assembly are part of a single unit. Also as seen, in an embodiment, the electrode array and the reservoir are part of a single unit.

In some embodiments, the teachings detailed herein enable an apparatus that is configured to deliver, on a first temporal period average, less than and/or equal to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, or 60 nanograms per hour or any value or range of values therebetween in 0.05 nanogram increments per hour of therapeutic substance during a first temporal period over a second temporal period without recharging. In an exemplary embodiment, the first temporal period is 1, 2, 3, 4, 5, 6, 7,8, 9, 10, 11, 12, 13, 14, 15, 16, 17, 18, 19, 20, 21, 22, 23, 24, 25, 26, 27, 28, 29, 30, 35, 40, 45, 50, 55, or 60 hours, or days, or any value or range of values therebetween in one hour or day increments, and the second temporal period is 14, 15, 16, 17, 18, 19, 20, 21, 22, 23, 24, 25, 26, 27, 28, 29, 30, 35, 40, 45, 50, 55, 60, 65, 70, 80, 90, 100, 125, 150, 175, 200, 250, 300 hours, or days, or any value or range of values therebetween in one hour or day increments. In an embodiment, the distinct barrier is both a bacterial filter and controls the rate of delivery/determines the rate of delivery of the therapeutic substance. Different barrier configurations (e.g., porosities) can be selected to obtain different diffusion rates. In an embodiment the barrier is of a design so that the barrier is relatively thin, sufficiently thin, so that it has relatively little impact on the diffusion rate. Other barriers may be more amenable to rate control in other embodiments. In an embodiment, to change/control a rate of delivery, a concentration in the reservoir can be changed/adjusted. For example, a high concentration can equate to a faster diffusion, at least initially, and visa-versa. Alternative and/or in addition to this, the cross-sectional area of the lengthy tubes (tubes 718 and/or 714) can be changed/modified to achieve a desired rate, at least approximately.

Also, in some embodiments, as detailed, the apparatus is configured such that the reservoir can be refilled or recharged without surgery.

Note that in an exemplary embodiment, there are sensors or the like that are included with the reservoir or otherwise with the electrode array assembly that can enable sensation or otherwise an evaluation of the amount of therapeutic substance that remains in the reservoir or otherwise in the electrode array assembly. In this regard, in an exemplary embodiment, the electrode array assembly can be configured to communicate a signal indicative or otherwise based on the level of the amount of therapeutic substance that can be communicated from the receiver-stimulator to the external component, thus giving an indication or otherwise enabling an indication of how much therapeutic substance remains or otherwise of any therapeutic substance remains, etc.

In view of the above, it is to be understood that in at least some exemplary embodiments, the reservoir is under pressure such that the drug is forced from the reservoir into the carrier body and/or the silicone body substantially limits the flow of drug out of the drug reservoir.

In an exemplary embodiment, the pressure under which the drug is located is a pressure that is no more than 1.01, 1.02, 1.03, 1.04, 1.05, 1,06, 1.07, 1.08, 1.09, 1.1, 1.12, 1.14, 1.16, 1.18, or 1.2 times greater than the ambient pressure inside the cochlea and/or the statistical average atmospheric pressure at sea level in Washington, D.C. for the calendar year 2021 based on data at Dulles Airport and/or 1 atmosphere or any value or range of values therebetween in increments of 0.01 times, all other things being equal. In this embodiment, the distinct barrier can be configured to prevent pressures above any one or more of the aforementioned pressures.

Any arrangement disclosed herein can be an arrangement that is refillable and/or rechargeable, unless otherwise specified. And again, other embodiments include implantable portions that cannot be re-filled or recharged, at least after implantation.

It is noted that any disclosure with respect to one or more embodiments detailed herein can be practiced in combination with any other disclosure with respect to one or more other embodiments detailed herein. That is, some exemplary embodiments include any one or more of the teachings detailed herein combined with any one or more of the other teachings detailed herein, unless otherwise stated such, providing that the art enables such. It is also noted that any disclosure herein of any feature corresponds to a disclosure of an exemplary embodiment that explicitly excludes that given feature from utilization with any one or more other features detailed herein unless otherwise specified providing that the art enables such.

It is noted that any disclosure herein of any method action corresponds to a disclosure of a device and/or system that enables that method action. It is noted that any disclosure herein of any method of manufacturing or otherwise developing or making a device disclosed herein corresponds to a disclosure of the resulting device that results from that method. It is noted that any disclosure herein of any apparatus and/or system corresponds to a disclosure of providing and/or making that apparatus and/or system. It is noted that any disclosure herein of any functionality corresponds to a device and/or system is configured to provide that functionality. It is noted that any disclosure of any device and/or system herein corresponds to a disclosure of a method of utilizing that device and/or system.

In this regard, it is noted that any disclosure of a device and/or system herein also corresponds to a disclosure of utilizing the device and/or system detailed herein, at least in a manner to exploit the functionality thereof. Further, it is noted that any disclosure of a method of manufacturing corresponds to a disclosure of a device and/or system resulting from that method of manufacturing. It is also noted that any disclosure of a device and/or system herein corresponds to a disclosure of manufacturing that device and/or system.

While various embodiments have been described above, it should be understood that they have been presented by way of example only, and not limitation. It will be apparent to persons skilled in the relevant art that various changes in form and detail can be made therein without departing from the spirit and scope of the invention. Thus, the breadth and scope of the present invention should not be limited by any of the above-described exemplary embodiments, but should be defined only in accordance with the following claims and their equivalents.

1. An apparatus, comprising:
an array of electrodes; and
an implantable therapeutic substance reservoir, wherein
the apparatus is an implantable portion of a cochlear implant,
the apparatus is configured so that the therapeutic substance reservoir extends from a location behind an ear canal of a human between a mastoid bone and skin of the human to the cochlea when the apparatus is fully implanted in a recipient, and
the reservoir is completely integrated into the implantable portion of the cochlear implant.

2. The apparatus of claim 1, wherein:
the reservoir has a volume of no more than 20 microliters when the implantable portion is implanted in the human.

3. The apparatus of claim 1, wherein:
the reservoir has a volume of no more than 10 microliters when the implantable portion is implanted in the human.

4. The apparatus of claim 1, wherein:
the reservoir has a volume of no more than 4 microliters when the implantable portion is implanted in the human.

5. An assembly, comprising:
the apparatus of claim 1, and
a reservoir fill assembly, wherein
the reservoir fill assembly is removably attached to the apparatus,
the reservoir fill assembly includes a septum configured to receive a termination of a syringe to enable a therapeutic substance to be delivered to the reservoir fill assembly and then into the reservoir.

6. (canceled)

7. The apparatus of claim 1, wherein:
the apparatus includes an integrated septum configured to receive a termination of a syringe to enable a therapeutic substance to be delivered to the reservoir, which integrated septum provides a bacterial seal between the reservoir and an outside environment of the apparatus after the termination is removed.

8. (canceled)

9. The apparatus of claim 1, wherein:
the apparatus is configured to removably receive a termination of a therapeutic substance charging device to enable therapeutic substance located in the charging device to be delivered to the reservoir; and
the apparatus is configured to seal the reservoir upon removal of the termination prior to implantation of the apparatus into a human so that the therapeutic substance will not escape the reservoir.

10. (canceled)

11. An apparatus, comprising:
an implantable therapeutic substance reservoir;
a plurality of implantable electrodes;
an implantable silicone carrier body supporting the plurality of electrodes; and
a stimulator assembly including an implantable housing and stimulation electronics, wherein
the therapeutic substance reservoir extends from the stimulator assembly to the silicone carrier body, and
the apparatus includes a fill port in fluid communication with the reservoir configured to enable therapeutic substance delivery to the reservoir, and the fill port is between the housing and the plurality of electrodes.

12. The apparatus of claim 11, wherein:
the fill port includes a resealable septum configured to receive a termination of a syringe, wherein the septum is configured provide a barrier to bacteria upon removal of the syringe from the septum after delivery of the therapeutic substance to the reservoir.

13. The apparatus of claim 12, wherein:
the resealable septum is above or under electrical lead wires extending from the stimulator assembly to the plurality of electrodes.

14. The apparatus of claim 11, wherein:
the apparatus is configured to deliver therapeutic substance to a cochlea from the electrode array in a totally passive manner with a steady state of pressure.

15. (canceled)

16. An assembly, comprising:
the apparatus of claim 11; and
a reservoir fill assembly in fluid communication with the reservoir via the fill port, wherein
the reservoir fill assembly is configured to be at least substantially filled with a therapeutic substance to fill the reservoir,
the reservoir fill assembly is removable from the fill port so that the apparatus can be implanted without the reservoir fill assembly.

17. The assembly of claim 11, wherein:
removal of the reservoir fill assembly reduces the volume of therapeutic substance accessible to the reservoir by at least 60%.

18-25. (canceled)

26. An apparatus, comprising:
an implantable electrode array including a plurality of electrodes supported by a silicone body, wherein
the electrode array includes a therapeutic substance delivery channel including at least one port, and
the port includes a distinct barrier made of a material that maintains a bacterial barrier prior to charging the channel with a water-based substance.

27. The apparatus of claim 26, wherein:
the distinct barrier is a plug.

28-29. (canceled)

30. The apparatus of claim 26. wherein:
the distinct barrier is made of polyvinylidene fluoride.

31. The apparatus of claim 26, wherein:
the distinct barrier is made of Durapore®.

32. The apparatus of claim 26, wherein:
the combination of the therapeutic substance delivery channel and/or the distinct barrier is configured to diffuse dexamethasone at a rate of no more than 10 nanograms/hour after implantation into a cochlea.

33. The apparatus of claim 26, wherein:
the combination of the therapeutic substance delivery channel and/or the distinct barrier is configured to diffuse dexamethasone at a rate of no more than 5 nanograms/hour after implantation into a cochlea.

34. The apparatus of claim 26, wherein:
the distinct barrier is also configured so that an over-pressure in the channel relative to an outside environment will cause dexamethasone to be driven out the distinct barrier while maintaining the distinct barrier for subsequent use as a diffusion plug.

35-36. (canceled)
